(19) United States
(12) Patent Application Publication
(10) Pub. No.: US 2025/0191180 A1
(43) Pub. Date: Jun. 12, 2025

(54) ARTIFICIAL INTELLIGENCE ARCHITECTURE FOR PREDICTING CANCER BIOMARKERS

(71) Applicant: The Regents of the University of California, Oakland, CA (US)

(72) Inventors: Scott Michael Lippman, La Jolla, CA (US); Erik Niels Bergstrom, La Jolla, CA (US); Ludmil Boyanov Alexandrov, San Diego, CA (US)

(21) Appl. No.: 18/844,925

(22) PCT Filed: Mar. 7, 2023
(86) PCT No.: PCT/US2023/063887

§ 371 Date: Sep. 6, 2024

Related U.S. Application Data
(60) Provisional application No. 63/269,033, filed on Mar. 8, 2022.
(60) Provisional application No. 63/483,237, filed on Feb. 3, 2023.

(51) Int. Cl. G06T 7/00; G16B 20/00; G16H 10/40; G16H 50/20

(52) U.S. Cl. CPC G06T 7/0012; G16B 20/00; G16H 10/40; G16H 50/20; G06T 2207/20016; G06T 2207/20081; G06T 2207/20084; G06T 2207/30024

(57) ABSTRACT

Methods and systems that pertain to predicting cancer biomarkers are disclosed. In some embodiments of the disclosed technology, a method of determining the presence of a biomarker of a biological sample includes providing a section of a biological sample, wherein the section of the biological sample has been treated with a stain, imaging one or more regions of the stained section of the biological sample at a first resolution and a second resolution thereby generating a first and second plurality of image data, reducing a parameter space of the first and second plurality of image data, thereby producing a reduced first and second plurality of image data, and determining the presence of a biomarker of the biological sample as an output of a trained predictive model when the trained predictive model is provided an input of the reduced first and second plurality of image data.

### CROSS-REFERENCE TO RELATED APPLICATION

This patent document claims priorities to and benefits of U.S. Provisional Patent Application No. 63/269,033, titled “ARTIFICIAL INTELLIGENCE ARCHITECTURE FOR PREDICTING CANCER BIOMARKERS” and filed on Mar. 8, 2022, and U.S. Provisional Patent Application No. 63/483,237, titled “ARTIFICIAL INTELLIGENCE ARCHITECTURE FOR PREDICTING CANCER BIOMARKERS” and filed on Feb. 3, 2023. The entire content of the aforementioned patent applications is incorporated by reference as part of the disclosure of this patent document.

### TECHNICAL FIELD

The disclosed technology relates to systems and methods for detecting clinically actionable biomarkers.

### BACKGROUND

Previous methods to detect clinically actional biomarkers for personalize cancer treatment rely almost exclusively on genomic sequencing or genotyping platforms, such as microarrays, targeted panel sequencing, whole-exome sequencing, or whole-genome sequencing Researchers are conducting a study to avoid traditional sequencing approaches.

### SUMMARY

Disclosed are materials, systems, devices and methods for predicting cancer biomarkers using a deep learning architecture.

In some implementations of the disclosed technology, a method of determining the presence of a biomarker in a biological sample includes obtaining a section of a biological sample, wherein the section of the biological sample has been treated with a stain, imaging one or more regions of the stained section of the biological sample at a first resolution and a second resolution to generate a first and second plurality of image data, reducing a parameter space of the first and second plurality of image data to produce a reduced first and second plurality of image data, and providing the first and the second plurality of image data to a trained predictive neural network and determining the presence of a biomarker in the biological sample as an output of the trained predictive neural network.

In some implementations of the disclosed technology, a method of generating a trained predictive model configured to determine a presence of a biomarker in a biological sample includes generating stained sections of one or more biological samples and corresponding biomarker labels, imaging one or more regions of the stained sections of the one or more biological samples at a first resolution and a second resolution to generate a first and second plurality of image data, reducing a parameter space of the first and second plurality of image data to produce a reduced first and second plurality of image data, and generating a trained predictive model, wherein the trained predictive model comprises a first predictive model trained with the reduced first plurality of image data and corresponding biomarker labels, and a second predictive model trained with the reduced second plurality of image data and corresponding biomarker labels.

In some implementations of the disclosed technology, a computer system configured to determine the presence of a biomarker of a biological sample includes one or more processors, and a non-transitory computer readable storage medium including software stored thereon, wherein the software comprises executable instructions that, as a result of execution, cause the one or more processors of the computer system to image one or more regions of a stained section of a biological sample at a first resolution and at a second resolution to generate a first and a second plurality of image data, reduce a parameter space of the first and second plurality of image data to produce a reduced first and second plurality of image data, and providing the first and the second plurality of image data to a trained predictive model and determine the presence of a biomarker in the biological sample as an output of the trained predictive model.

In some implementations of the disclosed technology, a method of determining the presence of a biomarker in a biological sample includes obtaining a stained section of the biological sample, imaging one or more regions of the stained section of the biological sample to generate a plurality of images of the stained section, and providing the plurality of images of the stained section an input to a trained predictive model and determining the presence of a biomarker in the biological sample as an output of a trained predictive model, wherein the trained predictive model is configured with a preset accuracy of determining the presence of the biomarker set to at least 80% of genomic sequencing.

In some implementations of the disclosed technology, a treatment method for treating cancer in a subject in need thereof includes obtaining a stained section of a biological sample, imaging one or more regions of the stained section of the biological sample to generate a plurality of images of the stained section, providing the plurality of images of the stained section to a trained predictive model and determining a presence of a biomarker in the biological sample as an output of the trained predictive model, wherein the trained predictive model is configured with a preset accuracy of determining the presence of the biomarker set to at least 80% of genomic sequencing, and administering treatment to the patient based on the presence of the biomarker.

The above and other aspects and implementations of the disclosed technology are described in more detail in the drawings, the description and the claims.

### BRIEF DESCRIPTION OF THE DRAWINGS

FIGS. 1A-1B show an example of multi-resolution convolutional neural network architecture to detect molecular biomarkers from histopathological tissue slides based on some implementations of the disclosed technology.

FIGS. 2A-2D show a neural network for detecting homologous recombination deficiency and predicting response to treatment in primary and metastatic breast cancer.

FIGS. 3A-3C show a transfer learning in ovarian cancer for predicting response to platinum treatment.

FIGS. 4A-4C show workflow for training neural network models independently for digitalized flash frozen and FFPE breast cancer slides.

FIGS. 5A-5B show workflow for testing the performance of neural network (e.g., DeepHRD) models for digitalized flash frozen and FFPE breast cancer slides.

FIG. 6 shows an example method of determining the presence of a biomarker of a biological sample based on some implementations of the disclosed technology.

FIG. 7 shows an example method of generating a trained predictive model configured to determine a presence of a biomarker of a biological sample based on some implementations of the disclosed technology.

FIG. 8 shows another example method of determining the presence of a biomarker of a biological sample based on some implementations of the disclosed technology.

FIG. 9 shows a treatment method for treating cancer in a subject in need thereof based on some implementations of the disclosed technology.

FIG. 10 shows an example of a computer system configured to determine the presence of a biomarker of a biological sample based on some implementations of the disclosed technology.

### DETAILED DESCRIPTION

The disclosed technology can be implemented in some embodiments to provide an artificial intelligence (AI) architecture platform for predicting cancer biomarkers, and provide therapeutic methods based on the biomarkers identified by the AI architecture platform.

The disclosed technology can be implemented in some embodiments to provide methods and systems for detecting clinically actionable cancer biomarkers and mutational signatures directly from digital hematoxylin and eosin (H&E) slides without sequencing.

The disclosed technology can also be implemented in some embodiments to provide a novel deep learning architecture that, with little to no customization, can be trained to predict clinically actionable molecular cancer biomarkers directly from digital images based on scans of slides stained using hematoxylin and eosin (H&E). The invention allows skipping DNA sequencing and provides direct prediction of these biomarkers from the scanned slides.

Previous methods to detect clinically actional biomarkers for personalize cancer treatment rely almost exclusively on genomic sequencing or genotyping platforms (e.g., microarrays, targeted panel sequencing, whole-exome sequencing, or whole-genome sequencing). The develop convolutional neural network architecture completely avoids traditional sequencing approaches by directly predicting clinically actional biomarkers using digital images from hematoxylin and eosin-stained (H&E) histology images sampled from individual patients. From a machine learning architecture implementation perspective, the approach utilizes semi-supervised convolutional neural networks to make segmented predictions within a grid space across whole-slide H&E images composed of three-color channels. The machine learning method aggregates these predictions to locate regions of interests and makes a final actional status prediction for each patient. Further, this model introduces a multi-resolution approach that captures morphological patterns at varying zoom magnifications and implements Monte Carlo dropout to provide confidence metrics that refine the final predictive value. Regions of interests are selected using an unsupervised machine learning module comprised of a dimensionality reduction using principal component analysis and custom k-means clustering algorithm on the extracted feature vectors for each component of the grid space. From an application perspective, the method requires training before it can be applied to a particular clinical biomarker. Specifically, the approach requires at least a thousand patients with digital H&E slides and known molecular biomarkers to generate a cancer-specific/biomarker-specific prediction model. After the model is trained, it can be applied to an individual patient making a prediction whether the cancer of the individual patient has the biomarker.

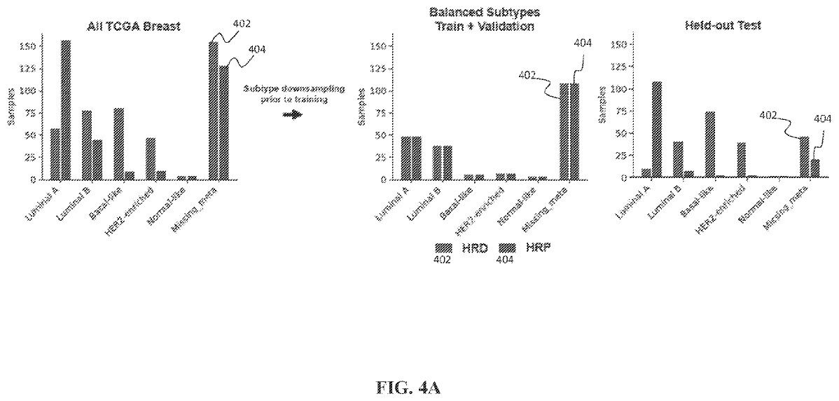

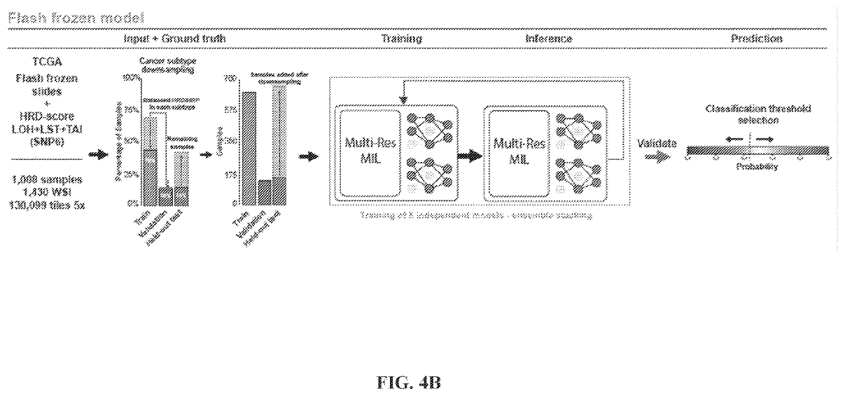

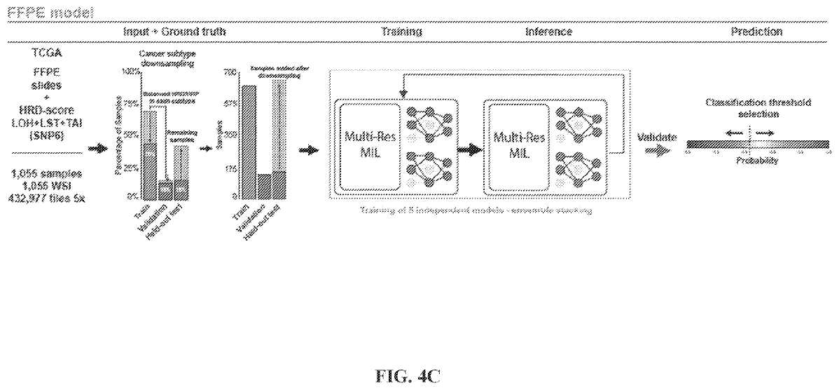

Hematoxylin and eosin (H&E) stain is one of the principal tissue stains used in histology. H&E slides are routinely and universally generated by pathologists for cancer diagnosis. However, in most cases, these slides do not allow pathologist to detect clinically actionable molecular biomarkers and do not provide guidance for personalized therapy. As such, in majority of cases, cancer samples are subsequently sent for DNA and/or RNA sequencing for detecting individual and/or sets of biomarkers. The novel deep learning architecture implemented based on some embodiments of the disclosed technology allows training an AI model that can directly predict biomarkers which are incorporated in therapeutic regimens thereby improving patient response to therapy and survival after patients are treated with therapy targeting the detected biomarker. The methods provided herein allow for identification of biomarkers directly from the digital H&E slides (see FIGS. 4A-4C), thus, skipping the need for shipping and sequencing of bio-specimens by external providers. For example, instead of sending a biospecimen over the mail to an external CLIA lab and waiting 14 days for results from sequencing, the AI approach allows directly detecting these biomarkers in the digital slides within a fraction of a second.

The disclosed technology can also be implemented in some embodiments to provide a novel deep learning architecture that, with little to no customization, can be trained to predict clinically actionable and/or epidemiologically relevant molecular signatures directly from digital images based on scans of slides stained using hematoxylin and eosin (H&E). The invention allows skipping DNA sequencing and provides direct prediction of these signatures from the digital images of the scanned slides. Previous methods to detect clinically actionable biomarkers for personalized cancer treatment or epidemiologically relevant biomarkers for large-scale genetic epidemiological studies relied almost exclusively on DNA sequencing or genotyping platforms (e.g., microarrays, targeted panel sequencing, whole-exome sequencing, and/or whole-genome sequencing). The developed convolutional neural network architecture completely avoids traditional sequencing approaches by accurately evaluating the presence or absence of molecular signatures using digital images from hematoxylin and eosin-stained histology images sampled from individual patients. The developed architecture requires at least 1,000 digital images of whole slides for training a model for a specifical molecular signature in a particular cancer type. Nevertheless, after a model is trained, accurate predictions can be made for a digital image from a single cancer patient.

From a machine learning and implementation perspective, the developed architecture utilizes semi-supervised convolutional neural networks to make segmented predictions within a grid space across whole-slide H&E images composed of three-color channels. The overall architecture is representative of a multi-resolution model that captures morphological patterns at two zoom magnifications with each magnification reflecting a separate convolutional neural network (CNN). Generally, the model performs initial predictions on a lower resolution (i.e., generally at 5× magnification) and automatically localizes regions of interest by identifying those with the highest predictive power. Subsequently, the model performs a secondary prediction at a higher resolution scale across the selected regions of interest (i.e., usually at 20× magnification). The resulting predictions are used in a final module that aggregates the scores across the multi-resolution model to provide a final prediction for a given molecular signature in a specific cancer type. The architectures little customization generally related to adjusting one of the two zoom levels (e.g., using 25× magnification for a generating a better model for a particular molecular signature).

The developed convolutional neural network uses a convolutional neural network architecture (e.g., ResNet) as its foundation with several important and significant modifications. In contrast, the newly developed convolutional neural network is trained using a binary cross entropy loss function based on the most predictive tile derived from each sample at a given magnification level. For example, digital image of a whole slide is tiled at 5× magnification and all sub-tiles are evaluated through an inference stage; the sub-tile(s) with highest predicted probability for each whole slide are used in a single training pass of the model. This process is repeated for each epoch throughout training of the model. Initially, this method aggregates the segmented predictions at a lower magnification to locate regions of interests. The automatic selection of the regions of interests is performed using an unsupervised machine learning module. The unsupervised machine learning module encompasses a dimension reduction algorithm based on principal component analysis of the extracted feature vectors for each component of the grid space, where the feature vectors are collected from the penultimate layer of the trained CNN and, subsequently, they are reduced to the two principal components contributing to the greatest variance across the collection of vectors. A custom k-means clustering module determines the most optimal number of clusters per sample by selecting the solution with maximum silhouette coefficients across all utilized iterations. The final regions of interests are chosen using the cluster encompassing the tile with the highest predictive value and including all other instances with silhouette coefficients within the top 50th percentile of the selected cluster. The complete set of selected tiles is subsequently used for training the second CNN model, where the CNN has an enhanced resolution (usually of 20×). The second enhanced resolution CNN is based on an identical architecture as the first CNN and it is trained solely on the regions of interest chosen in the first stage of the model. Each region of interest is resampled from the original whole-slide image at an increased magnification to capture more details at the cellular level. The proposed models were generally trained and tested after resampling at 20× magnification; however, the model can be used with any zoom preference. During inference, the tile with the highest predictive value is used to make the final prediction for a particular molecular signature across all regions of interest in a given sample. This enhanced predictive score is averaged with the predicted score from the first model to arrive at a final actionable status prediction for each patient.

Within both CNN models, random dropout of nodes within the fully connected layers has been incorporated to improve the robustness of the approach. The dropouts are utilized both during model training to overcome issues with overfitting as well as during apply the model for inference serving as a method to quantify a level of confidence for each patient-level prediction. Specifically, inference is run across a single whole-slide image over many iterations (at least 100 iterations but usually less than 1,000 iterations) with a new set of nodes randomly dropped with each pass of the model. This method acts as a Bayesian approximation of the underlying distribution of potential models for the developed architecture. Each pass of the model presents a single prediction score, and the resulting distribution of scores across all iterations for a single patient can be analyzed to determine the level of certainty with the final prediction. For example, a confident prediction is one with a low variance from the average predicted score, while an uncertain prediction will tend to have a high variance from the average predicted score. When applied to a single image of a whole slide for an individual patient, the developed architecture will provide a normalized prediction score between 1 (low) and 100 (high) and a confidence interval for the score.

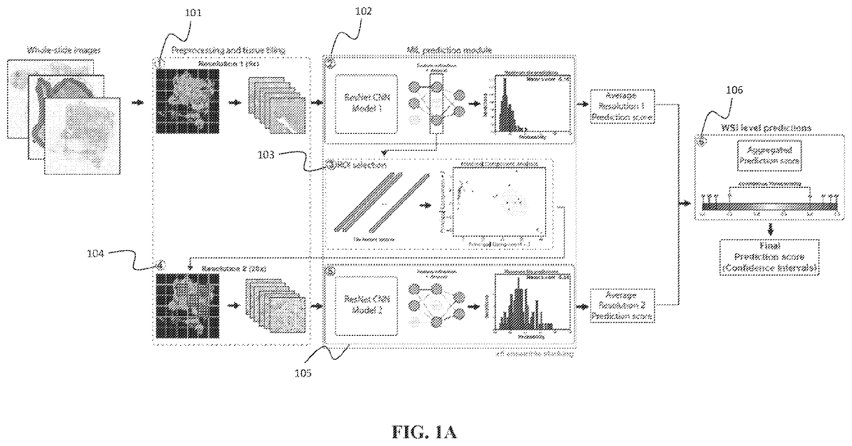

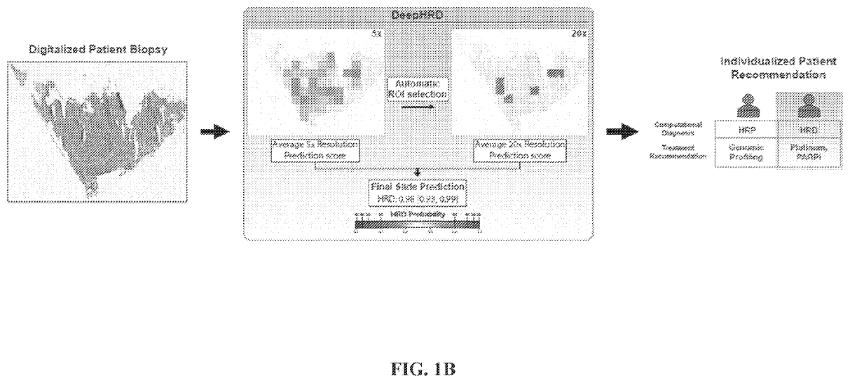

FIGS. 1A-1B show an example of multi-resolution convolutional neural network architecture to detect molecular biomarkers from histopathological tissue slides based on some implementations of the disclosed technology. As shown in FIGS. 1A-1B, the multi-resolution convolutional neural network architecture implemented based on some embodiments can detect homologous recombination deficiency from histopathological tissue slides. FIG. 1A shows training of a neural network (e.g., DeepHRD) model for detecting homologous recombination deficiency (HRD) from whole-slide images (WSIs). For each WSI, a single prediction score is estimated based on the detection of HRD. Specifically, at 101, each WSI undergoes preprocessing and quality control. This module consists of tissue segmentation, filtering for non-focused tissue, and final tiling of regions that contain tissue at 5× magnification. At 102, all tiles for a single image are processed through the first multiple instance learning (MIL) ResNet18 convolutional neural network. This architecture uses the average of the top 25 predicted tile scores as the WSI predicted score. Dropout is incorporated into the fully connected layers in the feature extraction module to reduce overfitting during training. The same dropout technique is also incorporated during inference to simulate Monte Carlo dropout used to calculate confidence intervals in the final WSI prediction. At 103, the tile feature vectors from the penultimate layer of the feature extraction are used to automatically select regions of interest (ROI) from the original WSI for additional assessment. The feature vectors are reduced in dimensions using principal component analysis and a custom k-means clustering module is used to determine the optimal number of clusters per sample. At 104, the selected tiles are then resampled at a 20× magnification. At 105, these sets of tiles are used to train a second MIL-ResNet18 model using an identical architecture to the one previously used in 102. At 106, the average predictions across both models are aggregated for a single WSI. The resulting distribution of scores are used to calculate confidence intervals and establish a threshold of confidence for a final prediction. FIG. 1B shows a trained neural network (e.g., DeepHRD) model for HRD prediction from a single whole-slide image. The trained neural network (e.g., DeepHRD) model produces a final prediction score for individual patient biopsies, with a computational-based diagnosis for subsequent clinical action.

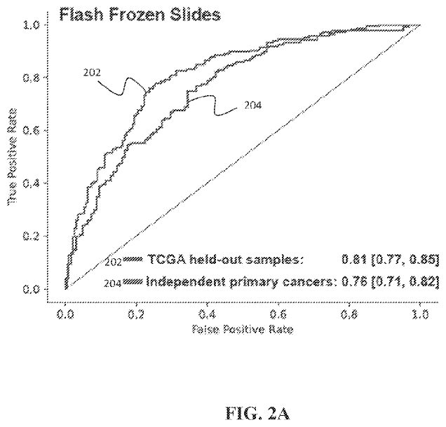

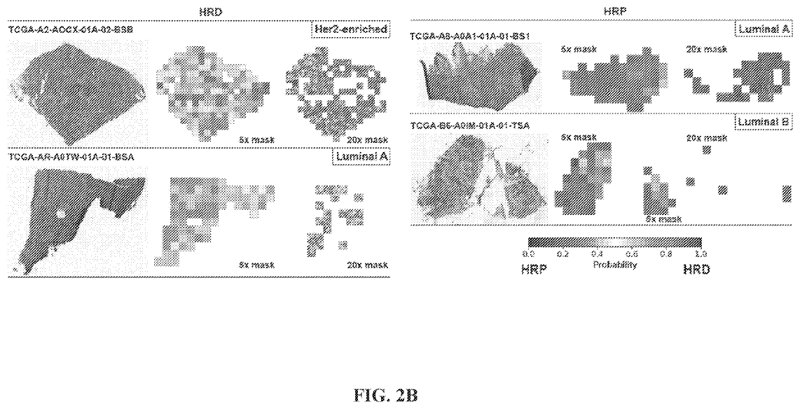

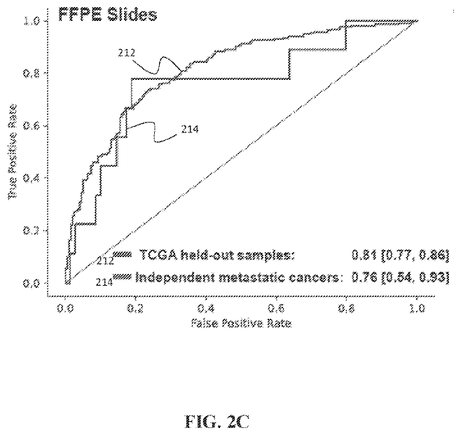

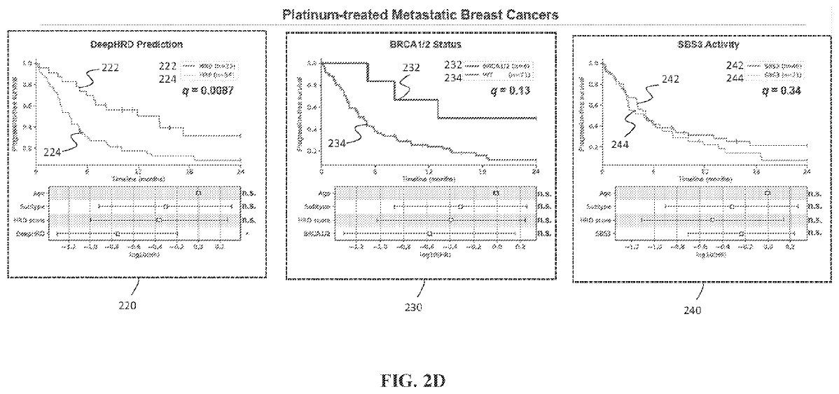

FIGS. 2A-2D show a neural network (e.g., DeepHRD) for detecting homologous recombination deficiency and predicting response to treatment in primary and metastatic breast cancer. FIG. 2A shows the receiver operating characteristic curves (ROCs) for classifying homologous recombination deficiency (HRD) in the TCGA held-out set (202) and the independent set (204) of primary breast cancers, encompassing the independent CPTAC and METABRIC primary breast cancer cohorts. FIG. 2B shows representative TCGA tissue slides are shown for both HRD and homologous recombination proficient (HRP) samples across multiple breast cancer subtypes along with the resulting predictions for each segmented tile at 5× and 20× resolutions. FIG. 2C shows ROCs for formalin-fixed paraffin-embedded (FFPE) diagnostic model in the TCGA held-out set (212) and for classifying metastatic breast cancer (MBC) patients who are complete responders to platinum therapy. FIG. 2D shows Kaplan-Meier survival curves for MBC patients treated with platinum chemotherapy separated by DeepHRD model predictions (220), BRCA1/2 mutation status (230), and SBS3 activity as predicted by SigMA (240). Q-values are corrected after considering breast cancer subtype, age at diagnosis, and the standard-of-care binary HRD classification score ≥42 (i.e., HRD score). Cox regression showing the log 10-transformed hazard ratios are shown with their 95% confidence intervals (bottom of 220, 230, 240). Q-values less than or equal to 0.05 are annotated with * while q-values above 0.05 are annotated with n.s. (i.e., non-significant).

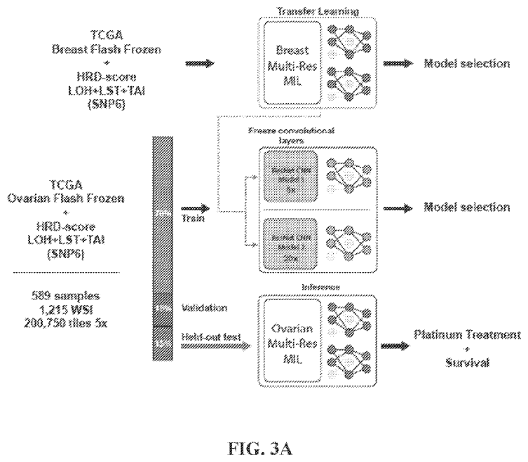

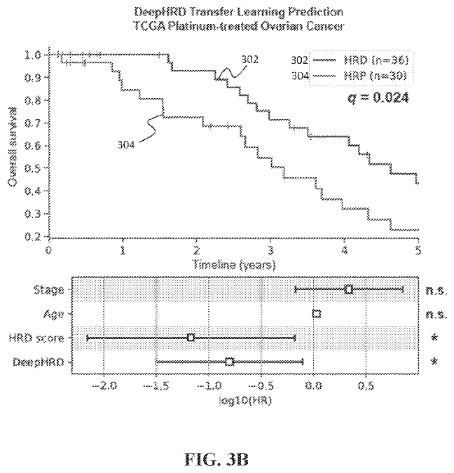

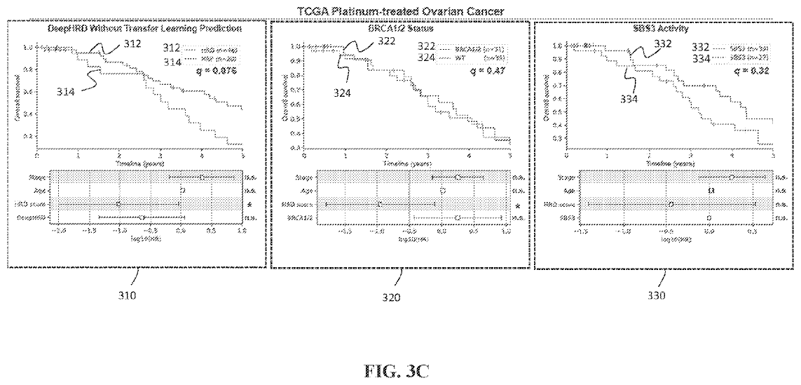

FIGS. 3A-3C show a transfer learning (e.g., DeepHRD transfer learning) in ovarian cancer for predicting response to platinum treatment. FIG. 3A shows schematic demonstrating the transfer learning method to train an ovarian homologous recombination deficiency (HRD) model from whole-slide H&E image (WSI) using a pretrained breast DeepHRD model. The pretrained flash-frozen breast model is used to initiate the weights and biases of all parameters in the ovarian model. HRD-scores are calculated from SNP6 genotyping microarray by deriving loss of heterozygosity (LOH), large-scale transitions (LST), and telomeric allelic imbalance (TAI). FIG. 3B shows Kaplan-Meier survival curves comparing the outcomes of patients treated with platinum chemotherapy split by the prediction of the DeepHRD transfer learning model. FIG. 3C shows Kaplan-Meier survival curves comparing the outcomes of platinum-treated patients split by the base model predictions with no transfer learning applied (310), BRCA1/2 mutation status (320), and SBS3 activity as predicted by SigMA (330). Q-values are corrected after considering ovarian cancer stage, age at diagnosis, and the standard-of-care binary HRD classification score ≥63 (i.e., HRD score). Cox regression showing the log 10-transformed hazard ratios are shown with their 95% confidence intervals (bottom of 310, 320, 330). Q-values less than or equal 0.05 are annotated with * while q-values above 0.05 are annotated with n.s. (i.e., non-significant).

FIGS. 4A-4C show workflow for training neural network (e.g., DeepHRD) models independently for digitalized flash frozen and FFPE breast cancer slides. FIG. 4A shows prior to training, the number of HRD and HRP samples within each breast cancer subtype were balanced using all available PAM50 annotations. FIGS. 4B and 4C show the collection of flash frozen (FIG. 4B) and formalin-fixed paraffin-embedded (FFPE) (FIG. 4C) slides for the TCGA breast cancer cohort were used to train two independent DeepHRD models. Prior to training, the number of HRD and HRP samples were balanced within each breast cancer subtype. All downsampled individuals were added to the internal held-out test set. The validation sets were used to optimize the classification thresholds.

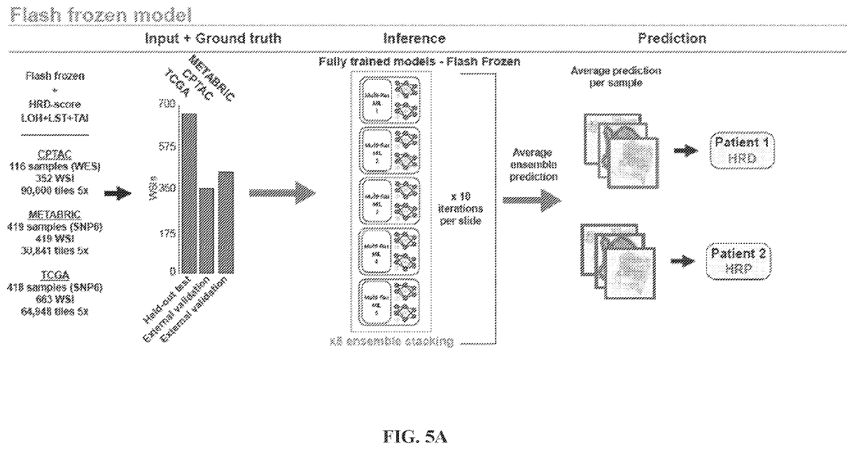

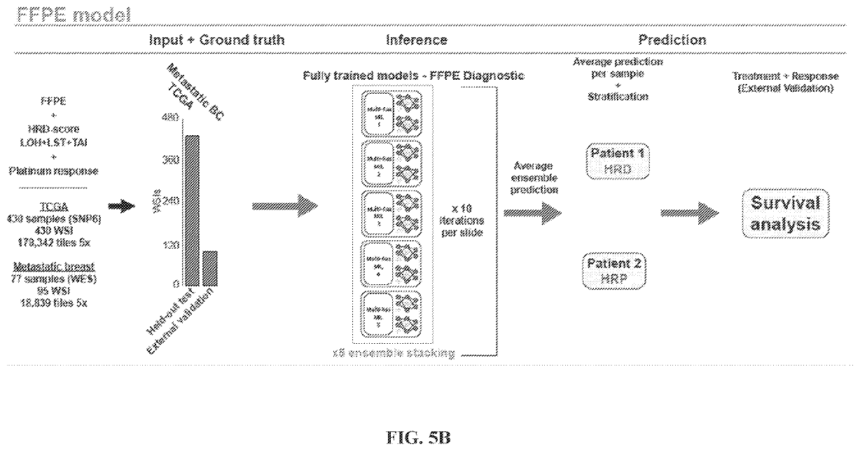

FIGS. 5A-5B show workflow for testing the performance of neural network (e.g., DeepHRD) models for digitalized flash frozen and FFPE breast cancer slides. FIG. 5A shows the collection of breast cancers from CPTAC and METABRIC were used to independently validate the flash frozen breast cancer model. The DeepHRD prediction scores were averaged for samples with multiple images. FIG. 5B shows an independent collection of metastatic breast cancers treated with platinum chemotherapy was used to validate the formalin-fixed paraffin-embedded (FFPE) breast cancer model based upon individual patient response to therapy.

The disclosed technology can be implemented in some embodiments to provide a deep learning artificial intelligence architecture that predicts genomic homologous recombination deficiency and platinum response from routine histology slides in breast and ovarian cancers.

Cancers harboring deficiencies in homologous recombination repair (HRD) can benefit from platinum-based chemotherapies and PARP inhibitors. Current standard diagnostic tests for detecting HRD in breast and ovarian cancers require genotyping-based or sequencing-based assays, which are not universally available.

The disclosed technology can be implemented in some embodiments to provide a novel multi-resolution deep learning approach that allows training robust models for detecting genomic biomarkers directly from digitalized images of hematoxylin and eosin (H&E)-stained light-microscopy histopathological slides. In some implementations, a model for predicting genomically derived HRD scores can be trained using a number of primary breast cancers (e.g., 1,008 primary breast cancers from the Cancer Genome Atlas (TCGA) project). In addition to a set of held-out TCGA samples, the trained breast cancer model was externally validated on 535 primary breast cancers from two independent research cohorts and on 77 platinum-treated metastatic breast cancers. Applicability to 589 TCGA ovarian tumors was also demonstrated by training and validating a model using transfer learning for predicting platinum response.

Across the TCGA breast cancer held-out validation cohort, the trained deep learning model primary breast cancers implemented based on some embodiments of the disclosed technology can predict genomically derived HRD scores from digital H&E slides with an AUC of 0.81 ([0.77-0.85] 95% Confidence Interval (CI)). This performance was confirmed in two independent primary breast cancer cohorts (AUC=0.76; [0.71-0.82] 95% CI). In an external clinical cohort of platinum-treated metastatic breast cancers, samples predicted by the deep learning model as HRD had a higher complete response (AUC=0.76; [0.54-0.93] 95% CI) and a 3.7-fold longer mean progression-free survival (hazard ratio=0.47; q=0.0087). Notably, the deep learning approach based on some embodiments of the disclosed technology can identify platinum-sensitive BRCA1/2 wild-type tumors as HRD-positive. By applying transfer learning, the approach based on some embodiments of the disclosed technology may also predict overall survival after first-line platinum treatment in advanced-stage, high-grade serous-type ovarian cancer (hazard ratio=0.45; q=0.024). The deep learning model implemented based on some embodiments of the disclosed technology can outperform multiple existing genomic HRD biomarkers within each cohort.

A deep learning model applied to digitalized H&E histopathological slides from breast and ovarian cancers detected genomically derived HRD and predicted direct clinical benefit to standard-of-care platinum-based therapies. The approach outperformed existing genetic biomarkers across multiple cohorts, slide scanners, and tissue-fixation procedures. These results have important implications for equitable and efficient clinical management of cancer patients sensitive to targeted DNA-damage-response therapies.

Precision oncology aims to personalize cancer therapy by first identifying and, subsequently, targeting molecular defects in tumors within each individual. Many cancers harbor failures of specific DNA repair pathways and utilizing synthetic lethal relationships amongst peripheral pathways has proven as an effective treatment approach. Specifically, exploiting treatments that increase DNA damage and/or provide inhibition of additional DNA repair pathways in cells with a pre-existing DNA repair defect can lead to selective cancer cell death. Previous mechanistic studies and clinical trials have shown that breast and ovarian cancers lacking the ability to repair DNA double strand breaks through homologous recombination are highly sensitive to DNA-damage-response targeted therapies like platinum treatment and Poly (ADP-ribose) polymerase (PARP) inhibitors.

Historically, homologous recombination deficiency (HRD) has been associated with germline mutations in specific genes leading to an increased risk for developing breast and ovarian cancers with the most notable susceptibility genes being BRCA1 and BRCA2. In addition to germline variants, somatic mutations and epigenetic dysregulation in breast and ovarian cancers have been shown to lead to HRD. Importantly, cancers deficient in homologous recombination exhibit genomic instability with characteristic patterns of somatic mutations and gene expression. Some of these patterns have also been leveraged for detecting HRD in the absence of canonical germline or somatic defects within HRD-associated genes. For instance, the pattern of single-base substitution signature 3 (SBS3), part of the Catalogue of Somatic Mutations in Cancer (COSMIC) catalog of mutational signatures, has been attributed to HRD independently of the molecular mechanisms disabling DNA repair through homologous recombination. Importantly, SBS3 has been previously utilized as a clinical biomarker for detecting HRD in breast and ovarian cancers.

In the United States, two commercial HRD companion diagnostic (CDx) tests have been approved by the U.S. Food and Drug Administration (FDA) for patients with ovarian and metastatic breast cancers. Myriad myChoice® CDx and Foundation One® CDx both determine HRD by quantifying overall genomic instability in combination with BRCA1 and BRCA2 status. Additionally, multiple researches and CLIA-certified diagnostic tests for detecting HRD have been developed by examining germline variants, somatic mutations, mutational patterns, changes in gene expression, and/or epigenetic modifications. While detection of homologous recombination deficiency can be performed using a multitude of different methods, all of these approaches intrinsically rely on assays profiling DNA and/or RNA leading to bottlenecks largely attributed to availability of molecular testing, time to decision making, and overall cost. In turn, this has precluded the widespread utilization of companion and complementary diagnostic biomarkers in standard therapy and CLIA-certified research testing for clinical trials. For example, the cost of a CLIA-certified companion for complementary HRD test is several thousand dollars making them unaffordable for many patients in the United States and most countries around the world. Furthermore, results from sequencing-based diagnostics can take 3 to 6 weeks, thus, severely delaying clinical management of many lethal and rapidly progressing solid tumors. Lastly, recent reports have demonstrated that only a small percentage of patients around the world have access to sequencing-based diagnostic tests, including FDA-approved companion diagnostic tests, with even lower testing rates in various underserved populations. This large ‘gap’ in cancer genomic testing presents a critical issue in the delivering equitable and efficient clinical management for all cancer patients worldwide necessitating the need for identifying low-cost scalable biomarkers for clinical oncology.

While the access and uptake of sequencing-based diagnostics is limited, tissue biopsies are routinely sampled and processed with hematoxylin and eosin staining (H&E) for solid-tumor diagnostics for most patients throughout the world. In combination with recent advances in computer vision and computational pathology for detecting recurring patterns in complex, data-rich whole-slide H&E images, deep learning artificial intelligence (AI)-based models allow for both prognostic and diagnostic predictions using only histopathological tissue slides. Here we introduce DeepHRD, a weakly-supervised multi-resolution convolutional neural network architecture for detecting HRD directly from digitalized H&E tissue slides. We train and validate DeepHRD models on data from The Cancer Genome Atlas (TCGA) project and demonstrate their ability to detect HRD using data from two external research consortia. Importantly, using independent clinical samples, we demonstrate that DeepHRD outperforms existing genomic biomarkers in predicting patient response to platinum-based therapies. By circumventing current bottlenecks in genomic testing, the method based on some embodiments of the disclosed technology has direct implications for addressing global socioeconomic disparities in the diagnosis and treatment of breast and ovarian cancers.

To train a downstream model capable of predicting HRD status directly from digitalized tissue slides, we implemented DeepHRD—a weakly supervised convolutional neural network architecture based upon the fundamental assumptions of multiple-instance learning (MIL; FIG. 1). We trained and internally validated our models using digitalized H&E images from the TCGA breast cancer cohort comprising 1,008 samples with flash frozen slides and 1,055 samples with formalin-fixed paraffin-embedded (FFPE) slides (FIGS. 4A-4C). We further trained a separate model using the TCGA ovarian cancer cohort comprising 589 samples with flash frozen slides. All included samples had accompanying whole-exome sequencing data as well as microarray genotyping data for calculating a genomic HRD score (FIGS. 4A-4C). The DeepHRD method was trained separately to predict HRD status from FFPE and flash frozen tissues of breast cancers as well as from flash frozen tissue of ovarian cancers resulting in a total of three independently trained DeepHRD models.

The flash frozen breast cancer model was externally validated using primary breast cancers from the: (i) Clinical Proteomic Tumor Analysis Consortium (CPTAC) comprised of 116 samples with associated whole-exome sequencing; and (ii) Molecular Taxonomy of Breast Cancer International Consortium (METABRIC) comprised of 419 samples with associated microarray genotyping data (FIG. 5A). The FFPE breast cancer model was externally validated in an independent clinical cohort of metastatic breast cancers comprised of 77 patients treated with platinum-based chemotherapy with available metastatic biopsies and associated genomic and clinical annotations. Clinical response to platinum-based therapy and progression-free survival (PFS) were assessed using the Response Evaluation Criteria in Solid Tumors, version 1.1 (RECIST 1.1; FIG. 5B). The collection of fixed tissue slides from the TCGA breast cancers, TCGA ovarian cancers, CPTAC cohort, and METABRIC cohort were digitalized using the Aperio ScanScope system. The clinical cohort of metastatic breast cancers was digitalized using the Hamamatsu Photonics Nanozoomer system.

### Definition of the Genomic HRD Score and Associated Genetic Markers

HRD scores were calculated from sequencing or genotyping data as previously reported using scarHRD. Briefly, scarHRD considers the combined aggregated score of the telomeric allelic imbalance score, loss of heterozygosity score, and large-scale transitions score calculated for each patient using ASCAT-derived copy number calls from SNP6 genotyping microarrays. Traditionally, an HRD score greater than 42 has been used to determine eligibility for treatment with platinum-based chemotherapy or PARP inhibitors in triple negative breast cancer, while an HRD score greater than 63 has been utilized for ovarian cancer. For our ground truth, we incorporated soft labelling during training to prevent the model from becoming overconfident with a single image by centering the HRD score cutoff at the median score across all breast cancer samples (i.e., HRD score cutoff of 30). All breast cancer samples with HRD scores above 50 were considered deficient, while all samples with HRD scores below 10 were considered proficient. The remaining breast cancers with HRD scores between 10 and 50 were modelled using soft labeling centered at 30 where there is an equal symmetric probability of a sample being deficient or proficient. For ovarian cancers, all ovarian samples with HRD scores above 73 were considered deficient, while all samples with HRD scores below 53 were considered proficient. Analogous to breast cancer, the remaining ovarian cancers with HRD scores between 53 and 73 were modelled using soft labeling centered at 63 where there is an equal symmetric probability of a sample being deficient or proficient.

The pathogenicity of mutations found within BRCA1 and BRCA2 was determined using InterVar as previously described for the TCGA ovarian cohort. All variants predicted as pathogenic were also considered deleterious. The mutational status of BRCA1 and BRCA2 within the metastatic breast cancer cohort was determined by screening variants across existing database annotations that included Clinvar, Swissprot, Leiden Open Variation Database (LOVD), and the Universal Mutations Database (UMD) as previously reported.

The activities of COSMIC mutational signature SBS3 has been shown to be enriched in HRD samples. The presence of SBS3 reflects many potential deficiencies that may arise across the HR machinery independent of the underlying mechanism of inactivation, which has more recently been used for detecting HRD from clinical sequencing data. To determine the presence or absence of SBS3 activity within the TCGA ovarian cohort and the metastatic breast cancer cohort, we used the high confidence predictions produced by the machine learning tool SigMA. SigMA classifies samples as either SBS3 positive or SBS3 negative with an estimated false positive rate (FPR) of 1% and a sensitivity of 50%.

The deep learning architecture implemented based on some embodiments of the disclosed technology can be built upon the concept of the weakly supervised MIL-assumptions (FIG. 1A). Specifically, all partitioned regions of a slide, or tiles, within a whole-slide H&E image (WSI) are assigned a weak label based upon the slide-level classification for each sample. It is assumed that all tiles within a negatively labeled slide are homologous recombination proficient (HRP), whereas at least a single tile must exhibit an HRD phenotype within a positively labeled slide. These assumptions allow the model to be trained using only a single classification label for an entire image without the need for detailed manual annotations from a pathologist, which currently does not exist for characterizing HRD.

The model is based on a multi-resolution decision, which performs an initial prediction on a low magnification (i.e., 5× magnification) and then automatically selects regions of interest (ROI) to perform a secondary prediction on an enhanced magnification within the selected ROIs (i.e., 20× magnification; FIG. 1A). DeepHRD's multi-resolution architecture framework was designed to mimic the standard diagnostic protocol used by pathologists to examine H&E images by first selecting ROIs at a low magnification across the entire tissue slide and then to refine the specific tumor characteristics and subtypes captured at a higher resolution. Further, DeepHRD maps individual tile predictions back to the original WSI, which allows visualizing the relative contribution and importance of specific tissue regions to the predictions of the model without using any pixel-level annotations (FIG. 1B). The final model encompasses an ensemble of five identical architectures, with each producing multi-resolution prediction scores. The average of these scores was used to make a final prediction for each tissue slide. Due to the computational cost associated with processing an entire WSI, each slide was first segmented into smaller tiles for each utilized resolution. For the first stage of the model, each slide was tiled at a 5× magnification with 256×256 pixels per tile with approximately 2 μm of tissue per pixel. Blurred tiles and those with less than 80% of pixels containing tissue were removed (FIG. 1A). For both stages of the model, ResNet18 convolutional neural networks were trained to extract features from the collection of tiles composing a single WSI. The resulting encoded features from the penultimate fully connected layer were used to automatically select ROIs at the 5× resolution. Specifically, principal component analysis (PCA) was used to project the encoded features into a latent space encompassing the greatest variance. K-means clustering was then used to group each tile representation. The total number of clusters was determined for each sample based upon the value of k that provided the maximum silhouette coefficient across all tile representations. The cluster containing the tile with the maximum prediction probability was selected along with all tiles in the same cluster having a silhouette score greater than the 95% quantile of all silhouette scores across the WSI. The final ROIs were tiled at 20× magnification (0.5 μm per pixel) and used to train and test the second model. The top 25 tiles were averaged to calculate a final prediction score at a given resolution during an inference pass of a WSI. Importantly, during training, random dropout of nodes within the fully connected layers of the ResNet architecture was incorporated to prevent overfitting of the training dataset. This same dropout technique, known as Monte Carlo dropout, was applied during inference of each WSI to provide an estimation of the model uncertainty by performing multiple inference passes over a single WSI. The resulting distribution of predictions were averaged to calculate a final score encompassing any epistemic uncertainty and were used to calculate confidence thresholds for a given sample (FIG. 1A).

Once the final HRD model has been trained and tuned, DeepHRD, can be used to make predictions on individual patients using only a digitalized cancer biopsy (FIG. 1B). Using a single WSI as input, DeepHRD will produce a prediction score with confidence intervals that are used to make a computational diagnostic recommendation. The intended use of this method is to provide a computational diagnosis for subsequent clinical action. Specifically, individuals with a high confidence prediction are labeled as either HRD or HRP (FIG. 1B).

To characterize the classification performance of the proposed methodology, we calculated the area under the receiving operating curve (AUROC or AUC). Confidence intervals were calculated using non-parametric resampling. Comparisons across survival curves were calculated using a log-rank test. Multivariate analyses were performed to calculate hazard ratios using Cox regressions. Corrections for multiple hypothesis testing were performed using the Benjamini Hochberg procedure.

A DeepHRD model was trained to detect HRD samples using a subset of flash frozen tissue slides from the TCGA breast cancer cohort (FIG. 1A). The TCGA breast cancer cohort was separated with: (i) 70% of samples used for training; (ii) 15% of samples used for validating the training and for adjusting prediction parameters; (iii) 15% of samples held-out and used for testing the final trained model (n=1,008 total breast cancer samples; FIGS. 4A-4C). Prior to training, the number of HRD and HRP samples in each breast cancer subtype were balanced to prevent the models from learning features specific to individual subtype histology rather than those directly associated with HRD (FIGS. 4A-4B). The trained DeepHRD models can then be applied to individual digital slides to provide a patient-level prediction revealing whether a breast cancer is HRD or HRP (FIG. 1B). Importantly, DeepHRD allows overlaying an HRD probability mask to each digital slide, which can be used for subsequent investigation into pathological characteristics of each breast cancer patient (FIG. 1B).

The final trained DeepHRD breast cancer model was then tested on the held-out TCGA test set to assess the overall performance resulting in an AUC of 0.81 ([0.77-0.85] 95% Confidence Interval (CI); FIG. 2A). To assess the generalizability of the model, we performed an external validation using the collection of breast cancer slides from CPTAC and METABRIC (FIG. 5A) resulting in an AUC of 0.76 ([0.71-0.82] 95% CI; FIG. 2A). Importantly, while HRD is enriched in luminal B, basal-like, and Her2 enriched breast cancers (FIG. 4A), the model implemented based on some embodiments of the disclosed technology can distinguish HR deficiency and proficiency across all subtypes (FIG. 2B).

While flash frozen tissue slides are commonly used for downstream molecular analyses, FFPE tissue slides are standard in clinical settings. Therefore, we trained an independent model to classify HRD directly from FFPE slides from the TCGA breast cancer cohort following the same procedure as previously described for training on flash frozen tissue images (FIGS. 4A-4C). The final TCGA FFPE model exhibited an AUC of 0.81 ([0.77-0.86] 95% CI; FIG. 2C), which was identical to the TCGA flash frozen model. These results indicate that the fixation procedure and differences in staining coloration have minimal effects on the performance of predicting HRD status directly from breast cancer tissue slides.

Importantly, the FFPE model was capable of distinguishing metastatic breast cancer (MBC) samples, part of an independent clinical cohort, that had a complete response to platinum chemotherapy (n=9) from those having only a partial or no response to treatment (n=68) resulting in an AUC of 0.76 ([0.54-0.93] 95% CI; FIG. 2C; FIG. 5B). Separating the MBC samples treated with platinum based upon DeepHRD's prediction revealed a differential clinical benefit between the HRD and HRP predicted samples with a median progression-free survival of 14.4 months for HRD patients and 3.9 months for HRP patients (p-value=0.0019, log-rank test). The model's predictive value was consistent after correcting for breast cancer subtype, age of diagnosis, and the genomic HRD score with a hazard ratio of 0.47 ([0.27-0.83] 95% CI, q-value=0.0087; Cox Proportional Hazards regression; FIG. 2D). Further, DeepHRD captured 7 of the 9 samples that had a complete response to platinum treatment. In comparison, neither the separation of samples based upon the genomic status of BRCA1/BRCA2 nor using the elevated activity of the mutational signature associated with HRD (SBS3 as predicted by SigMA) resulted in a significant difference in progression-free survival (q-value=0.13 and q=0.34, respectively; Cox Proportional Hazards regression; FIG. 2D). While the small sample size of BRCA1/2 mutated tumors (˜8% of the examined MBC samples) influenced the significance levels compared to wild-type tumors, the predictions from DeepHRD captured 4-fold more platinum sensitive samples than using BRCA1/2 status alone. Lastly, the tissue slides from the MBC cohort were digitalized using the Hamamatsu Nanozoomer system, while the TCGA breast cancers were digitalized using the Aperio Scan Scope system demonstrating the generalizability of DeepHRD across different scanning protocols.

To further assess the generalizability of the proposed method in application to other cancers, we performed transfer learning on the TCGA ovarian cancer cohort (FIG. 3A). Individuals with ovarian cancer have traditionally received platinum chemotherapies as the first-line standard of care making this cohort ideal to test whether HRD predictions from tissue slides may have a direct clinical benefit. Specifically, we trained an independent model to predict HRD status from flash frozen slides using the TCGA ovarian cohort. Due to a two times smaller cohort size (n=589 ovarian cancers), the ovarian model was initiated using the pretrained weights and biases generated from the flash frozen breast cancer model with the convolutional weights and biases frozen during training (FIG. 3A). The final model was applied to the held-out test set of TCGA ovarian cancers to assess the ability of the model in separating individuals who benefit from treatment with platinum chemotherapy (FIG. 3A). Of the 117 patients in the held-out set, 66 received first-line platinum chemotherapy for advanced-stage, high-grade serous ovarian cancer. Separating these individuals by their DeepHRD prediction resulted in a differential median survival between HRD and HRP predicted patients (FIG. 3B). Specifically, patients predicted to be HRD had a median survival of 4.6 years, while those predicted to be HRP had a median survival of 3.2 years (q-value=0.024) with a hazard ratio of 0.45 after correcting for the stage of the cancer, age, and the genomic HRD score ([0.22-0 90] 95% CI; Cox Proportional Hazards ratio; FIG. 3B). In comparison, we observed a worse separation of samples when using a base model without transfer learning (q-value=0.076) with a hazard ratio of 0.53 after corrections ([0.26-1.07] 95% CI; Cox Proportional Hazards ratio; FIG. 3C), suggesting that the transfer learning provides a benefit when attempting to train AI-based approaches on smaller datasets. Consistent with the breast cancer cohort, neither separation of samples based upon the mutational status of BRCA1/BRCA2 nor based on the elevated activity of SBS3 resulted in a significant difference in survival (q-value=0.47 and q-value=0.32, respectively; Cox Proportional Hazards regression; FIG. 3C).

The development of DeepHRD prediction models for breast and ovarian cancers demonstrates the practicality of employing AI-based guidance into clinical diagnostics and precision medicine workflows. Results across multiple publicly available (TCGA, CPTAC, and METABRIC) and additional external cohorts indicate that the models are applicable to routinely sampled tissue blocks and are generalizable across different cancers, histological and molecular subtypes, digital scanning systems, and tissue fixation procedures, including variability in H&E tissue staining. The performance of DeepHRD was consistent across primary and metastatic breast cancers and, by incorporating transfer learning, the model was also applicable to serous ovarian cancer. Most importantly, based on RECIST 1.1 criteria, DeepHRD predicted clinical response and progression-free survival to DNA-damage-response targeted therapies, namely, platinum therapies, and outperformed existing genomic based diagnostic biomarkers (viz., BRCA1/2 status and signature SBS3). Furthermore, consistent with prior breast cancer genomic studies, DeepHRD captured patients with BRCA1/2 wild-type tumors who responded to platinum therapy, identifying 4-fold more responsive patients than BRCA1/2 mutation-testing alone (FIG. 2D). These results demonstrate that genotyping-based and sequencing-based assays, traditionally used for assessing HRD in a clinical setting, can be substituted and/or complemented with AI-based deep learning models that can rapidly predict clinical response from routine digitalized diagnostic histopathological slides. Circumventing the reliance on genomic profiling provides a solution which is more readily deployable into the clinic while delivering greater accessibility to state-of-the-art diagnostics for a larger proportion of the population across diverse socioeconomic groups.

Historically, diagnosis of solid cancers has evolved from microscopic morphological assessment of H&E slides to genomic biomarker testing. While crucial for clinical management of certain cancers, genomic testing has substantially complicated routine clinical oncology workflows as it often requires re-biopsy to procure tumor tissue sufficient for molecular assays as well as extensive analytics to analyze the large-scale data generated by these molecular assays. Recent deep learning AI approaches has demonstrated the ability to detect genomic biomarkers directly from H&E images, including ones indirectly related to therapy outcome (e.g., detection of microsatellite instability that can be predictive of response to immunotherapy). In some implementations, no prior study has shown direct clinical significance of AI-based models for detecting HRD by predicting treatment benefit with external validations. Since HRD is a complementary biomarker to help guide the use of platinum therapies and an FDA-approved companion diagnostic test for the use of PARP inhibitors, the performance of the neural network (e.g., DeepHRD) implemented based on some embodiments of the disclosed technology has direct implications for predicting response to DNA-damage-response targeting therapies within breast, ovarian, and other cancer types with known HR-deficiencies. One such example is pancreatic ductal adenocarcinoma (PDAC), where patients with HRD detected using an FDA-approved targeted-sequencing assay had an improved clinical outcome with standard first-line platinum-based treatment. A severe limitation to using genomic detection of HRD in PDAC standard clinical practice was that, in addition to issues with tissue procurement for genomic studies, the 3-6 week turn-around for obtaining molecular profiling was not appropriate for first-line treatment of advanced disease due to a median survival of only 3-6 months with rapid progression in some PDAC patients. For such cancer types, AI-based detection of HRD from routinely generated H&E slides may provide a better and faster diagnostic alternative.

While there has been an explosion of deep learning and computer vision-based approaches in digital pathology, the immediate translation into clinical practice has been limited by the lack of global accessibility in developing countries and resource-constrained communities due to the required infrastructure and the high overhead costs associated with streamlining digital pathology. Nevertheless, a recent study has shown the potential for deploying deep learning models trained on whole slide images directly to hand-held photographs of core-needle-biopsied tissues taken from a microscope's field of view. These types of digitalized tissue images are smaller in size and ultimately require a tenth of the computational resources to process for downstream diagnostics making them readily deployable on a local smartphone device with a standard high-resolution camera attached to the ocular lens of a conventional light microscope. This approach promises inexpensive, efficient, and accurate deep-learning read-outs within seconds of preparing an H&E slide. In coordination with the development of lightweight deep learning architectures, there are opportunities for deploying diagnostic applications, which are traditionally computationally expensive, into a manageable package without a substantial decrease in predictive power. By relying on smartphone microscopy images, this transition would provide AI-based diagnostic solutions for equitable and efficient clinical management for all cancer patients worldwide.

### Additional Methods

### Data Sources

The collection of flash frozen and formalin-fixed paraffin-embedded (FFPE) slides from TCGA along with all clinical features were downloaded from the Genomic Data Commons (GDC; https://gdc.cancer.gov/). The collection of flash frozen slides from CPTAC were downloaded from The Cancer Imaging Archive (TCIA), and the genomics data was downloaded from the GDC. The collection of images from METABRIC and the associated SNP6 genotyping microarray data were downloaded from EGA (accession numbers: EGAD00010000270 and EGAD00010000266). The predicted cancer subtype for a subset of the TCGA breast cancer cohort were obtained from a previous study that utilized the 50-gene PAM50 model. HRD scores for the TCGA breast and ovarian cancers were obtained from a previous study. The 77 patients from the clinical cohort of whole-exome sequenced metastatic breast cancers were enrolled between June 2018 and March 2020 and all received at least one line of platinum chemotherapy. All clinical evaluations were determined locally at the Georges Francois Leclerc Cancer Center as previously reported.

### Data Preprocessing

Each of the whole-slide images (WSIs) was segmented into 256×256 tiles at 5× and 20× magnifications containing 2 μm per pixel and 0.5 μm per pixel, respectively. Blurry tiles and those with less than 80% of pixels representing tissue were removed from all training and testing cohorts. To filter blurry tiles, a Laplacian filter was applied to each tile using a 3×3 kernel, and all tiles with a variance less than 0.02 were removed from the remaining analysis. All green, red, and blue pen marks and other annotation artifacts were removed by thresholding on the RGB color channels within each pixel.

### Calculating HRD Scores

HRD scores were calculated as previously reported using scarHRD. Specifically, the HRD score is the summation of the telomeric allelic imbalance score, loss of heterozygosity score, and large-scale transitions scores calculated for each patient using ASCAT-derived copy number calls from SNP6 genotyping microarrays. The HRD scores for the CPTAC breast cancer samples were calculated based on copy number calls derived from whole-exome sequencing using Sequenza, which has been shown to result in analogous distributions of HRD scores to HRD scores calculated using ASCAT-derived copy number calls from SNP6 genotyping microarrays.

For both TCGA breast and ovarian cancer cohorts, soft labeling was applied to the HRD scores using specified thresholds for samples labeled confidently as HRD or HRP. These thresholds were determined by first splitting samples within each cancer type into HRD and HRP partitions using a single cutoff (HRD>=30 for breast and HRD>=63 for ovarian). The median values of the two resulting partitions of samples for each cancer type were used to set the range of confident HRD and HRP thresholds. All intermediate HRD scores were modeled as a probability using a quadratic function (equation 1).

$${HRD}_{Adjust} = \qquad ( 1 )$$

$$\{ \begin{matrix}\begin{matrix}2 * {( \frac{{HRD}_{thresh} - x}{{HRD}_{thresh} - {HRP}_{thresh}} - 0.5 )}^{2} + 0.5 \\ \end{matrix} & \begin{matrix}if {HRD}_{median} \leq \\ x < {HRD}_{thresh}\end{matrix} \\ \begin{matrix}1 - [ 2 * {( \frac{{HRD}_{thresh} - x}{{HRD}_{thresh} - {HRP}_{thresh}} - 0.5 )}^{2} + 0.5 ] \\ \end{matrix} & \begin{matrix}if {HRP}_{thresh} \leq \\ x < {HRD}_{median}\end{matrix}\end{matrix}$$

Specifically, HRD scores above 50 were considered HR-deficient and scores below 10 were considered proficient in the breast cancer cohorts. All intermediate scores were modelled as a probability of being deficient or proficient with an equal probability of both conditions at an HRD score of 30 (equation 2).

$$HR {D}_{Adjust} = \{ \begin{matrix}2 * {( \frac{50 - x}{50 - 10} - 0.5 )}^{2} + 0.5 & if 30 \leq x < 50 ( breast ) \\ 1 - [ 2 * {( \frac{50 - x}{50 - 10} - 0.5 )}^{2} + 0.5 ] & if 10 \leq x < 30 ( breast )\end{matrix} \qquad ( 2 )$$

Within the TCGA ovarian cohort, HRD scores above 73 were considered deficient and scores below 53 were considered proficient with the intermediate probabilities centered at 63 (equation 3).

$${HRD}_{Adjust} = \{ \begin{matrix}2 * {( \frac{73 - x}{73 - 53} - 0.5 )}^{2} + 0.5 & if 63 \leq x < 73 ( ovarian ) \\ 1 - [ 2 * {( \frac{73 - x}{73 - 53} - 0.5 )}^{2} + 0.5 ] & if 53 \leq x < 63 ( ovarian )\end{matrix} \qquad ( 3 )$$

### Model Training and Testing

Prior to training, the number of HRD and HRP samples were balanced in all breast cancer subtypes using the PAM50 model classifications to normalize for specific breast cancer subtypes being enriched or depleted of HRD samples. All samples without annotated PAM50 subtype labels were considered as missing and were also balanced for the number of HRD and HRP cases (FIG. 4A). Soft labelling was incorporated to prevent overfitting during training and to account for ambiguity in the ability of the HRD score to classify true HRD samples. The entirety of training and testing was performed using the machine learning Python framework Pytorch (v.1.5.0). For both resolution models, the Adam optimizer was used for training with a learning rate of 10⁻³, a weight decay of 10⁻⁴, and minibatches consisting of 64 tiles. Each model was initiated using the ResNet18 architecture that was pretrained on the ImageNet (http://www.image-net.org/) database and was trained for 200 epochs. All convolutional weights were frozen during training. Early stoppage was incorporated to prevent overfitting.

After training the 5× resolution models, a final inference pass is performed on all slides. All features from a single WSI were selected from the penultimate layer of the feature extractor and projected into a lower dimensional latent space using principal component analysis. K-means clustering was used to automatically select regions of interests (ROIs) for retiling at 20× magnification. The number of clusters was determined by selecting the solution with the maximum silhouette coefficient. The cluster containing the tile with the highest prediction probability was used to select the ROIs. All tiles belonging to this cluster, and which had a silhouette score greater than the 95% quantile of all silhouette scores for the given WSI were chosen as the final ROIs. Each ROI was then tiled into 256×256 pixel sub-tiles at 20× magnification. This results in 16 tiles at 20× magnification for each ROI at a 5× magnification. To perform an inference pass of the model, a single WSI image is processed across 10 iterations with a random dropout probability of 0.20 for all nodes within the fully connected layers.

### Transfer Learning

The weights collected from the final models trained to detect HRD from flash frozen breast slides were used to initiate the model weights for the ovarian model known as transfer learning. The held-out internal validation set was used to perform survival analysis based upon prior treatment with platinum chemotherapy. There were not enough FFPE slides for the ovarian cohort for training and testing a DeepHRD model for FFPE ovarian cancer samples.

### Visualizing DeepHRD Predictions

Once successfully trained, DeepHRD is used to make predictions for individual whole-slide images. When performing the multi-resolution inference, DeepHRD generates HRD probabilities for each tile at 5× magnification and for each tile within the automatically selected regions of interest at 20× magnification. Using the location of the original tiles, the probabilities can be mapped back to the original location within the whole-slide image to visualize the regional patterns that are influencing the final model prediction.

### Survival Analysis

Survival analysis was performed using the Lifelines Python package (v.0.24.4.). For both the metastatic breast cancer (MBC) and the TCGA ovarian cohorts, samples were partitioned based upon the prediction from each respective DeepHRD model. Only samples that were treated with platinum chemotherapy were considered in the survival comparisons. Survival curves were compared using a log-rank test. Hazard ratios were calculated from Cox regressions after correcting for age of diagnosis, primary breast cancer subtype, and genomic HRD score within the MBC cohort and age of diagnosis, ovarian cancer stage, and genomic HRD score within the TCGA ovarian cohort. Median survival was calculated as the time at which the chance of surviving beyond that point is 50%.

### Statistics

All performance metrics were calculated using the scikit-learn Python package (v.0.22.1). Confidence intervals were calculated using non-parametric resampling. Standard error bars were calculated using the NumPy Python package (v.1.18.1).

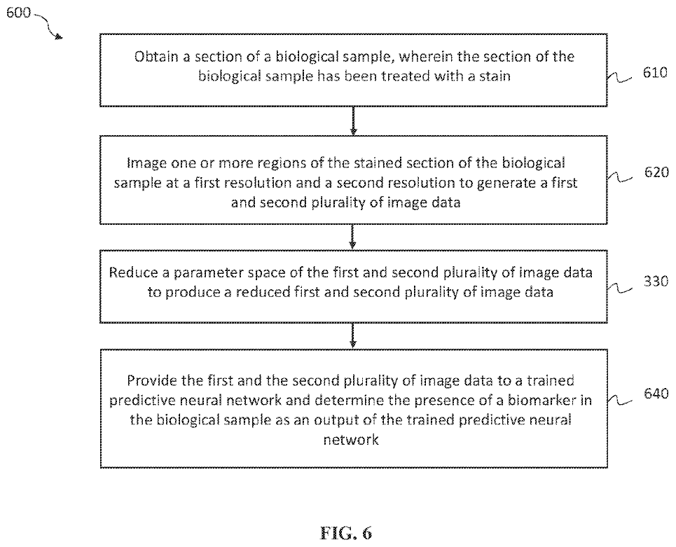

FIG. 6 shows an example method 600 of determining the presence of a biomarker in a biological sample based on some implementations of the disclosed technology.

In some implementations of the disclosed technology, the method 600 may include, at 610, obtaining a section of a biological sample, wherein the section of the biological sample has been treated with a stain, at 620, imaging one or more regions of the stained section of the biological sample at a first resolution and a second resolution to generate a first and second plurality of image data, at 630, reducing a parameter space of the first and second plurality of image data to produce a reduced first and second plurality of image data, and at 640, providing the first and the second plurality of image data to a trained predictive neural network and determining the presence of a biomarker in the biological sample as an output of the trained predictive neural network.

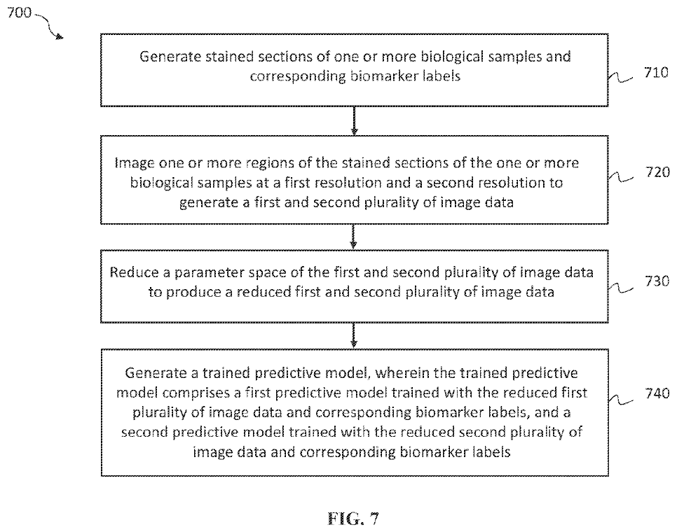

FIG. 7 shows an example method 700 of generating a trained predictive model configured to determine a presence of a biomarker in a biological sample based on some implementations of the disclosed technology.

In some implementations of the disclosed technology, the method 700 may include, at 710, generating stained sections of one or more biological samples and corresponding biomarker labels, at 720, imaging one or more regions of the stained sections of the one or more biological samples at a first resolution and a second resolution to generate a first and second plurality of image data, at 730, reducing a parameter space of the first and second plurality of image data to produce a reduced first and second plurality of image data, and at 740, generating a trained predictive model, wherein the trained predictive model comprises a first predictive model trained with the reduced first plurality of image data and corresponding biomarker labels, and a second predictive model trained with the reduced second plurality of image data and corresponding biomarker labels.

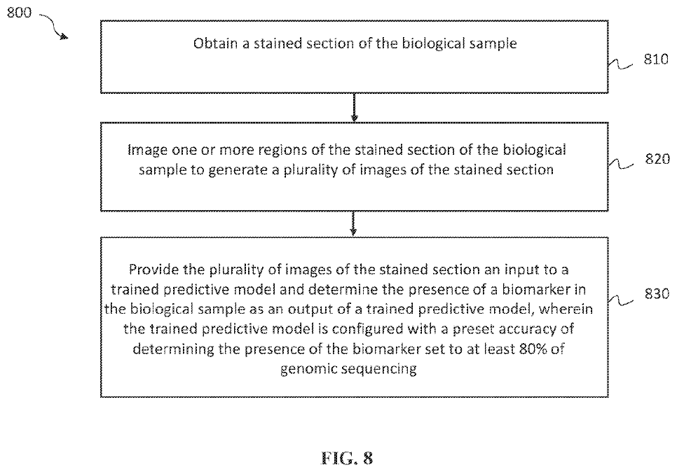

FIG. 8 shows another example method 800 of determining the presence of a biomarker of a biological sample based on some implementations of the disclosed technology.

In some implementations of the disclosed technology, the method 800 may include, at 810, obtaining a stained section of the biological sample, at 820, imaging one or more regions of the stained section of the biological sample to generate a plurality of images of the stained section, and at 830, providing the plurality of images of the stained section an input to a trained predictive model and determining the presence of a biomarker in the biological sample as an output of a trained predictive model, wherein the trained predictive model is configured with a preset accuracy of determining the presence of the biomarker set to at least 80% of genomic sequencing.

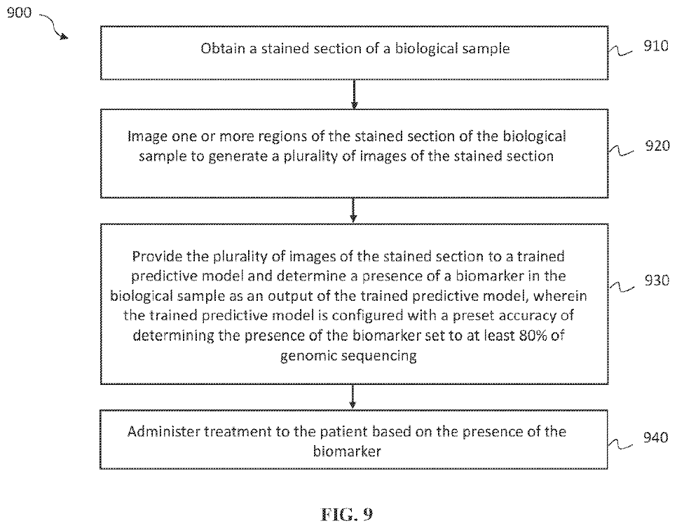

FIG. 9 shows a treatment method 900 for treating cancer in a subject in need thereof based on some implementations of the disclosed technology.

In some implementations of the disclosed technology, the method 900 may include, at 910, obtaining a stained section of a biological sample, at 920, imaging one or more regions of the stained section of the biological sample to generate a plurality of images of the stained section, at 930, providing the plurality of images of the stained section to a trained predictive model and determining a presence of a biomarker in the biological sample as an output of the trained predictive model, wherein the trained predictive model is configured with a preset accuracy of determining the presence of the biomarker set to at least 80% of genomic sequencing, and at 940, administering treatment to the patient based on the presence of the biomarker.

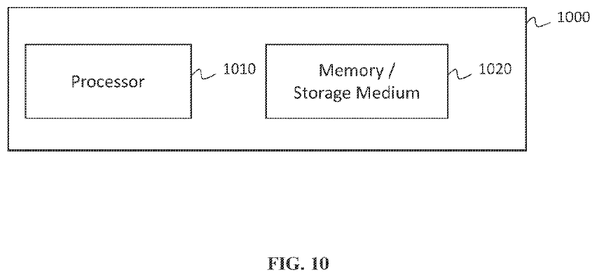

FIG. 10 shows an example of a computer system 1000 configured to determine the presence of a biomarker of a biological sample based on some implementations of the disclosed technology.

In some implementations of the disclosed technology, the system 1000 includes a processor 1010 and a memory or storage medium 1020. The processor 1010 reads code from the memory 1020 and implements a method discussed in this patent document.

Therefore, various implementations of features of the disclosed technology can be made based on the above disclosure, including the examples listed below.

### Method of Utilizing a Trained Predictive Model to Determine the Presence of a Biomarker in a Section

Example 1. A method of determining the presence of a biomarker of a biological sample, comprising: (a) providing a section of a biological sample, wherein the section of the biological sample has been treated with a stain; (b) imaging one or more regions of the stained section of the biological sample at a first resolution and a second resolution thereby generating a first and second plurality of image data; (c) reducing a parameter space of the first and second plurality of image data, thereby producing a reduced first and second plurality of image data; and (d) determining the presence of a biomarker of the biological sample as an output of a trained predictive model when the trained predictive model is provided an input of the reduced first and second plurality of image data.

Example 2. The method of example 1, wherein the trained predictive model is configured to determine the presence of the biomarker with an accuracy of at least 80% as compared to genomic sequencing.

Example 3. The method of example 2, wherein the accuracy comprises at least 85%, at least 92%, at least 95%, at least 97%, or at least 99% as compared to genomic sequencing.

Example 4. The method of example 1, wherein the trained predictive model comprises a first predictive model trained on the first plurality of image data and a second predictive model trained on the second plurality of image data.

Example 5. The method of example 1, wherein the biomarker comprises loss of chromosome 9p.

Example 6. The method of example 1, wherein the biomarker comprises presence of clustered mutations in the gene TP53. In some implementations, TP53 include a tumor suppressor gene. In one example, TP53 indicates tumor protein P53.

Example 7. The method of example 1, wherein the biomarker comprises presence of clustered mutations in the gene EGFR (epidermal growth factor receptor).

Example 8. The method of example 1, wherein the biomarker comprises presence of clustered mutations in the gene BRAF. In some implementations, BRAF includes a human gene that encodes a protein called B-Raf. In one example, BRAF indicates v-raf murine sarcoma viral oncogene homolog B1.

Example 9. The method of example 1, wherein the biomarker comprises presence of clustered mutations in the gene KIT.

Example 10. The method of example 1, wherein the biomarker comprises presence of MSI (vs MSS) and/or MMR gene (e.g., POLE, MLH1, MLH3, MGMT, MSH6, MSH3, MSH2, PMS1, or PMS2) defects. In some implementations, the MSI gene defect indicates a microsatellite instable (MSI) gene defect, and the MMR gene defect indicates a mismatch repair gene defect. In some implementations, the MSS indicates microsatellite stable.

Example 11. The method of example 1, wherein the biomarker comprises presence of high tumor mutational burden.

Example 12. The method of example 1, wherein the biomarker comprises presence of hypermutator mutational signatures selected from: POLE comprised of POLE and MSI-COSMIC14 (POLE+MSI); MSI combined MSI-COSMIC15, MSI-COSMIC20 (POLD+MSI), MSI-COSMIC21, MSI-COSMIC26, and MSI-COSMIC6.

Example 13. The method of example 1, wherein the biomarker comprises presence of apolipoprotein B mRNA editing enzyme, catalytic polypeptide (APOBEC) alterations and mutational signature. In some implementations, APOBEC indicates a family of evolutionarily conserved cytidine deaminases.

Example 14. The method of example 1, wherein the biomarker comprises presence of homologous recombination deficiency (HRD). Two commercial HRD companion diagnostic (CDx) tests, Myriad myChoice® CDx and FoundationOne® CDx, have been FDA approved to determine HRD by quantifying overall genomic instability in combination with BRCA1 and BRCA2 status, and, at least three academic HRD detection approaches (SigMA, HRDetect, and CHORD) exist. In some implementations, BRCA indicates breast cancer gene.

Example 15. The method of example 1, wherein the biomarker comprises presence of HRD negative (or homologous recombination proficiency, HRP) or HRD positive, for example, using genomic tests in Example 14.

Example 16. The method of example 1, wherein the biomarker comprises presence of BRCA1/2 mutations.

Example 17. The method of example 1, wherein the biomarker comprises presence of “COSMIC3-BRCA” mutational signature, comprising a specific pattern of genome-wide somatic single nucleotide variations (SNVs) defined as “mutational signature 3” (Sig3) in the COSMIC signature catalog or the presence of genomic ‘scar’ signatures.

Example 18. The method of example 1, wherein the biomarker comprises presence of genomic instability score (GIS), comprised of patterns (or signatures) of loss of heterozygosity (LOH); number of telomeric imbalances (telomeric allelic imbalance, or TAI), which are the number of regions with allelic imbalance that extend to the sub-telomere but not across the centromere; and large-scale state transitions (LST), which are chromosome breaks (deletions, translocations, and inversions).

Example 19. The method of example 1, wherein the biomarker comprises presence of a homologous recombination feature set which comprises: a total number and proportions of deletions at microhomologies features of the sequencing data, a total number and proportions of genomic segments with loss of heterozygosity features of the sequencing data, a total number and proportions of heterozygous genomic segments features of the sequencing data, a total number and proportions of C:G>T:A single base substitutions at a 5′-NpCpG-3′ contexts features of the sequencing data, or any combination thereof.

Example 20. The method of example 1, wherein the biomarker comprises presence of genomic alterations in one or more of the following homologous recombination repair (HRR)-related or -associated genes beyond BRCA1, BRCA2 (also called ‘BRCA-ness’): alterations in PALB2, ATM, ATR, CHEK1/2, FANC genes (FANCA/C/D2/E/F/G/I/L/M/1), RAD50, RAD51 genes (RAD51 B/C/D/L1/3), RAD52, RAD54L/C/D/B, ATRX, BAP1, BARD1, BRIP1, CDK12, PPP2R2A, MRE11, MRE11 A, NBN, TP53, NCOR1, PTK2, ARIDIA, BLM, WRN, CDK12, RPA1, EMSY, CCNE1, ERCC3, TAD54, XRCC2/3, HDAC2.

Example 21. The method of example 1, wherein the biomarker comprises presence of potentially actionable genomic alterations in one or more of the following genes: ABL1, AKT1, ALK, APC, ATM, BRAF, RET, ROS, KRAS, NRAS, HRAS, RAF1, IDH1, IDH2, JAK1, JAK2, JAK3, KDR, KIT, MAP2K1, MET, NTRK, NTRK1, CCNE, CCNE1, CDK4/6, CCND1/2, AR, PDGFRA, PIK3CA, PTEN, CDH1, CDKN2A, CSFIR, CTNNB1, DDR2, DNMT3A, EGFR, ERBB2, ERBB3, ERBB4, HER2/NEU, EZH2, FBXW7, FGF, FGFR, FGFR1, FGFR2, FGFR3, FLT3, FOXL2, GNA11, GNAQ, GNAS, HNFIA, MLH1, MPL, MSH6, NOTCH1, VEGFA, HGF, NPM1, PTPN11, RB1, SMAD4, SMARCB1, SMO, SRC, STK11, TP53, TSC1, VHL, ESR1, MAPK3K1, GATA3, CDH1, FBXW7, NF1, KMT2C, CTNNB1, GNA13, GNAQ, GNA11, RRAS2, KIFIA, KIF5B.

Example 22. The method of example 1, wherein the biomarker indicates copy number alterations, deletions, amplifications, fusions, mutation clusters, mutation signatures or any combination thereof the genome of the biological sample.

Example 23. The method of example 1, wherein the section of the biological sample comprises a paraffin embedded section, a formalin fixed section, a frozen section, a fresh section, or any combination thereof sections.

Example 24. The method of example 1, wherein the trained predictive model comprises a convolutional neural network.

Example 25. The method of example 1, wherein the trained predictive model comprises a neural network such as ResNet model.

Example 26. The method of example 1, further comprising reducing a parameter space of the first and second plurality of image data, thereby producing a reduced first and second plurality of image data. In some implementations, the parameter space of the first and second plurality of image data indicates tiles at 5× magnification, wherein the parameter space of the first and second plurality of image data is reduced to 25%, 10%, or 5% of the tiles carrying predictive information.

Example 27. The method of example 26, wherein reducing is completed by principal component analysis.

Example 28. The method of example 1, wherein the biological sample comprises a cancer free, or cancerous biological sample.

Example 29. The method of example 1, wherein the biological sample comprises healthy tissue, unhealthy tissue, or any combination thereof tissues.

Example 30. The method of example 29, wherein the unhealthy tissue comprises virally infected tissue.

Example 31. The method of example 30, wherein virally infected tissue comprises human papilloma virus (HPV) positive tissue.

Example 32. The method of example 30, wherein the virally infected tissue comprises Epstein-Barr virus (EBV), Hepatitis B virus (HBV), Hepatitis C virus (HCV), Human immunodeficiency virus (HIV), Human herpes virus 8 (HHV-8), and/or Human T-cell leukemia virus type, also called human T-lymphotrophic virus (HTLV-1).

Example 33. The method of example 29, wherein the unhealthy tissue comprises nuclei morphology different from nuclei morphology of healthy tissue.

Example 34. The method of example 33, wherein the unhealthy tissue comprises premalignant or precancerous tissue.

Example 35. The method of example 1, wherein the stain comprises a hematoxylin and eosin stain.

Example 36. The method of example 1, wherein the first resolution comprises a 5× magnification, and wherein the second resolution comprises a 20× magnification.

Example 37. The method of example 26, further comprising clustering the reduced first and second plurality of image data thereby generating a first and second clustered dataset.

Example 38. The method of example 37, wherein clustering is completed by k-means clustering.

Example 39. The method of example 37, wherein the trained predictive model is trained with the clustered datasets that represent the top 15% of the variance between clustered datasets of the first and second clustered datasets and corresponding biomarker labels.

Example 40. The method of example 37, wherein the trained predictive model is trained with the first and second clustered dataset and corresponding biomarker label of the biological sample, wherein the first and second clustered dataset comprise clustered datasets with silhouette coefficients within the top 50th percentile across all clusters of the first and second clustered dataset.

Example 41. The method of example 40, wherein the corresponding biomarker label of the biological sample is determined by genomic sequencing.

Example 42. The method of example 1, wherein the output of the trained predictive model comprises an averaged predicted probability score of the first and second predictive model.

Example 43. The method of example 1, wherein the one or more regions comprise at least 100 regions.

Example 44. The method of example 1, wherein the one or more regions comprise at most 10,000 regions.

Example 45. The method of example 1, comprising removing one or more nodes of the trained predictive model when the trained predictive model is provided an input of the reduced first and second plurality of image data.

### Method of Training a Predictive Model

Example 46. A method of generating a trained predictive model configured to determine a presence of a biomarker of a biological sample, comprising: (a) providing stained sections of one or more biological samples and corresponding biomarker labels; (b) imaging one or more regions of the stained sections of the one or more biological samples at a first resolution and a second resolution thereby generating a first and second plurality of image data; (c) reducing a parameter space of the first and second plurality of image data, thereby producing a reduced first and second plurality of image data; and (d) generating a trained predictive model, wherein the trained predictive model comprises a first predictive model trained with the reduced first plurality of image data and corresponding biomarker labels, and a second predictive model trained with the reduced second plurality of image data and corresponding biomarker labels.

Example 47. The method of example 46, wherein the trained predictive model is configured to determine the presence of a biomarker with an accuracy of at least 80% as compared to genomic sequencing.

Example 48. The method of example 47, wherein the accuracy comprises at least 85%, at least 92%, at least 95%, at least 97%, or at least 99% as compared to genomic sequencing.

Example 49. The method of example 46, wherein the biomarker label comprises loss of chromosome 9p.

Example 50. The method of example 46, wherein the biomarker comprises presence of clustered mutations in the gene TP53.

Example 51. The method of example 46, wherein the biomarker comprises presence of clustered mutations in the gene EGFR.

Example 52. The method of example 46, wherein the biomarker comprises presence of clustered mutations in the gene BRAF.

Example 53. The method of example 46, wherein the biomarker comprises presence of clustered mutations in the gene KIT.

Example 54. The method of example 46, wherein the biomarker comprises presence of MSI (vs MSS) and/or MMR gene defects comprising one or more of POLE, MLH1, MLH3, MGMT, MSH6, MSH3, MSH2, PMS1, and PMS2.

Example 55. The method of example 46, wherein the biomarker comprises presence of hypermutator mutational signatures selected from POLE, MSI-COSMIC14, (POLE+MSI); MSI combined, MSI-COSMIC15, MSI-COSMIC20, (POLD+MSI), MSI-COSMIC21, MSI-COSMIC26, and MSI-COSMIC6.

Example 56. The method of example 46, wherein the biomarker comprises presence of APOBEC alterations and mutational signature.

Example 57. The method of example 45, wherein the biomarker comprises presence of high tumor mutational burden.

Example 58. The method of example 46, wherein the biomarker comprises presence of homologous recombination deficiency (HRD). Two commercial HRD companion diagnostic (CDx) tests, Myriad myChoice® CDx and FoundationOne® CDx, have been FDA approved to determine HRD by quantifying overall genomic instability in combination with BRCA1 and BRCA2 status, and, at least three academic HRD detection approaches—SigMA, HRDetect, and CHORD—exist.

Example 59. The method of example 46, wherein the biomarker comprises presence of HRD negative (or homologous recombination proficiency, HRP) or HRD positive, for example, using genomic tests in Example 14.

Example 60. The method of example 46, wherein the biomarker comprises presence of BRCA 1/2 mutations.

Example 61. The method of example 46, wherein the biomarker comprises presence of “COSMIC3-BRCA” mutational signature, comprising a specific pattern of genome-wide somatic single nucleotide variations (SNVs) comprising “mutational signature 3” (Sig3) in the COSMIC signature catalog, or the presence of genomic ‘scar’ signatures.

Example 62. The method of example 46, wherein the biomarker comprises presence of genomic instability score (GIS), comprised of patterns (or signatures) of loss of heterozygosity (LOH), which are regions of intermediate size (over 15 MB and less than the whole chromosome); number of telomeric imbalances (telomeric allelic imbalance, or TAI), which are the number of regions with allelic imbalance that extend to the sub-telomere but not across the centromere; and large-scale state transitions (LST), which are chromosome breaks (deletions, translocations, and inversions).

Example 63. The method of example 46, wherein the biomarker comprises presence of a homologous recombination feature set which comprises: a total number and proportions of deletions at microhomologies features of the sequencing data, a total number and proportions of genomic segments with loss of heterozygosity features of the sequencing data, a total number and proportions of heterozygous genomic segments features of the sequencing data, a total number and proportions of C:G>T:A single base substitutions at a 5′-NpCpG-3′ contexts features of the sequencing data, or any combination thereof.

Example 64. The method of example 46, wherein the biomarker comprises presence of genomic alterations in one or more of the following homologous recombination repair (HRR)-related or -associated genes beyond BRCA1, BRCA2 (also called ‘BRCA-ness’): alterations in PALB2, ATM, ATR, CHEK1/2, FANC genes (FANCA/C/D2/E/F/G/I/L/M/1), RAD50, RAD51 genes (RAD51 B/C/D/L1/3), RAD52, RAD54L/C/D/B, ATRX, BAP1, BARD1, BRIP1, CDK12, PPP2R2A, MRE11, MRE11 A, NBN, TP53, NCOR1, PTK2, ARIDIA, BLM, WRN, CDK12, RPA1, EMSY, CCNE1, ERCC3, TAD54, XRCC2/3, HDAC2.

Example 65. The method of example 46, wherein the biomarker comprises presence of potentially actionable genomic alterations in one or more of the following genes: ABL1, AKT1, ALK, APC, ATM, BRAF, RET, ROS, KRAS, NRAS, HRAS, RAF1, IDH1, IDH2, JAK1, JAK2, JAK3, KDR, KIT, MAP2K1, MET, NTRK, NTRK1, CCNE, CCNE1, CDK4/6, CCND1/2, AR, PDGFRA, PIK3CA, PTEN, CDH1, CDKN2A, CSFIR, CTNNB1, DDR2, DNMT3A, EGFR, ERBB2, ERBB3, ERBB4, HER2/NEU, EZH2, FBXW7, FGF, FGFR, FGFR1, FGFR2, FGFR3, FLT3, FOXL2, GNA11, GNAQ, GNAS, HNFIA, MLH1, MPL, MSH6, NOTCH1, VEGFA, HGF, NPM1, PTPN11, RB1, SMAD4, SMARCB1, SMO, SRC, STK11, TP53, TSC1, VHL, ESR1, MAPK3K1, GATA3, CDH1, FBXW7, NF1, KMT2C, CTNNB1, GNA13, GNAQ, GNA11, RRAS2, KIFIA, KIF5B/SEE RK MSKCC LINK, DS/CARIS EMAIL.

Example 66. The method of example 46, wherein the stained sections of the one or more biological samples comprises paraffin embedded sections, formalin fixed sections, frozen sections, fresh sections, or any combination thereof sections.

Example 67. The method of example 46, wherein the trained predictive model comprises a convolutional neural network.

Example 68. The method of example 46, wherein the trained predictive model comprises a ResNet model.

Example 69. The method of example 46, wherein reducing is completed by principal component analysis.

Example 70. The method of example 46, wherein the one or more biological samples comprise a cancer free, cancerous biological sample, healthy tissue, unhealthy tissue, or any combination of health and unhealthy tissues.

Example 71. The method of example 70, wherein the unhealthy tissue comprises virally infected tissue.

Example 72. The method of example 71, wherein the virally infected tissue comprises human papilloma virus (HPV) positive tissue.

Example 73. The method of example 46, wherein the virally infected tissue comprises Epstein-Barr virus (EBV), Hepatitis B virus (HBV), Hepatitis C virus (HCV), Human immunodeficiency virus (HIV), Human herpes virus 8 (HHV-8), and/or Human T-cell leukemia virus type, also called human T-lymphotrophic virus (HTLV-1).

Example 74. The method of example 70, wherein the unhealthy tissue comprises nuclei morphology different from nuclei morphology of healthy tissue.

Example 75. The method of example 74, wherein the unhealthy tissue comprises premalignant or precancerous tissue.

Example 76. The method of example 46, wherein the stain comprises a hematoxylin and eosin stain.

Example 77. The method of example 46, wherein the first resolution comprises a 5× magnification, and wherein the second resolution comprises a 20× magnification.

Example 78. The method of example 46, further comprising clustering the reduced first and second plurality of image data thereby generating a first and second clustered dataset.

Example 79. The method of example 78, wherein clustering is completed by k-means clustering.

Example 80. The method of example 78, wherein the trained predictive model is trained with clustered datasets that represent the top 15% of the variance between clustered datasets of the first and second clustered datasets and corresponding biomarker labels.

Example 81. The method of example 78, wherein the first and second predictive models are trained with one or more biological samples' first and second clustered dataset and the corresponding biomarker labels, wherein the first and second clustered dataset comprise clustered datasets with silhouette coefficients within the top 50th percentile across all clusters of the first and second clustered dataset.

Example 82. The method of example 46, wherein the corresponding biomarker labels of the one or more biological samples are determined by genomic sequencing.

Example 83. The method of example 46, wherein the output of the trained predictive model comprises an averaged predicted probability score of the first and second predictive model.

Example 84. The method of example 46, wherein the one or more regions comprise at least 100 regions.

Example 85. The method of example 46, wherein the one or more regions comprise at most 1,000 regions.

Example 86. The method of example 46, wherein generating the trained predictive model comprises removing one or more nodes of the first and second predictive model during training.

### System Using Trained Predictive Model to Determine the Presence of a Biomarker of a Biological Sample

Example 87. A computer system configured to determine the presence of a biomarker of a biological sample, comprising: one or more processors; and a non-transient computer readable storage medium including software, wherein the software comprises executable instructions that, as a result of execution, cause the one or more processors of the computer system to: (i) receive a section of a biological sample, wherein the section of the biological sample has been stained; (ii) image one or more regions of the stained section at a first resolution and a second resolution thereby generating a first and second plurality of image data; (iii) reduce a parameter space of the first and second plurality of image data, thereby producing a reduced first and second plurality of image data; and (iv) determine the presence of a biomarker of the biological sample as an output of a trained predictive model when the trained predictive model is provided an input of the reduced first and second plurality of image data.

Example 88. The system of example 87, wherein the trained predictive model is configured to determine the presence of the biomarker with an accuracy of at least 80% as compared to genomic sequencing.

Example 89. The system of example 88, wherein the accuracy comprises at least 85%, at least 92%, at least 95%, at least 97%, or at least 99% as compared to genomic sequencing.

Example 90. The system of example 87, wherein the trained predictive model comprises a first predictive model trained on the first plurality of image data and a second predictive model trained on the second plurality of image data.

Example 91. The system of example 87, wherein the biomarker comprises loss of chromosome 9p.

Example 92. The system of example 87, wherein the biomarker comprises presence of clustered mutations in the gene TP53.

Example 93. The system of example 87, wherein the biomarker comprises presence of clustered mutations in the gene EGFR.

Example 94. The system of example 87, wherein the biomarker comprises presence of clustered mutations in the gene BRAF.

Example 95. The system of example 87, wherein the section of the biological sample comprises a paraffin embedded section, a formalin fixed section, a frozen section, a fresh section, or any combination thereof sections.

Example 96. The system of example 87, wherein the trained predictive model comprises a convolutional neural network.

Example 97. The system of example 87, wherein the trained predictive model comprises a ResNet model.

Example 98. The system of example 87, wherein reducing is completed by principal component analysis.

Example 99. The system of example 87, wherein the biological sample comprises healthy tissue, unhealthy tissue, or any combination thereof tissues.

Example 100. The system of example 99, wherein the unhealthy tissue comprises virally infected tissue.

Example 101. The system of example 100, wherein the virally infected tissue comprises human papilloma virus (HPV) positive tissue.

Example 102. The system of example 100, wherein the virally infected tissue comprises Epstein-Barr virus (EBV), Hepatitis B virus (HBV), Hepatitis C virus (HCV), Human immunodeficiency virus (HIV), Human herpes virus 8 (HHV-8), and/or Human T-cell leukemia virus type, also called human T-lymphotrophic virus (HTLV-1).

Example 103. The system of example 99, wherein the unhealthy tissue comprises nuclei morphology different from nuclei morphology of healthy tissue.

Example 104. The system of example 99, wherein the unhealthy tissue comprises premalignant or precancerous tissue.

Example 105. The system of example 87, wherein the biological sample comprises a cancer free, or cancerous biological sample.

Example 106. The system of example 87, wherein the stain comprises a hematoxylin and eosin stain.

Example 107. The system of example 87, wherein the first resolution comprises a 5× magnification, and wherein the second resolution comprises a 20× magnification.

Example 108. The system of example 87, wherein the instructions further comprise cluster the reduced first and second plurality of image data thereby generating a first and second clustered dataset.

Example 109. The system of example 108, wherein the instruction of clustering is completed by k-means clustering.

Example 110. The system of example 108, wherein the trained predictive model is trained with the clustered datasets that represent the top 15% of the variance between clustered datasets of the first and second clustered datasets and corresponding biomarker labels.

Example 111. The system of example 108, wherein the trained predictive model is trained with the biological sample's first and second clustered dataset and corresponding biomarker labels of the biological samples, wherein the first and second clustered dataset comprise clustered datasets with silhouette coefficients within the top 50th percentile across all clusters of the first and second clustered dataset.

Example 112. The system of example 111, wherein the corresponding biomarker label of the biological sample is determined by genomic sequencing.

Example 113. The system of example 87, wherein the output of the trained predictive model comprises an averaged predicted probability score of the first and second predictive model.

Example 114. The system of example 87, wherein the one or more regions comprise at least 100 regions, or at most 1,000 regions, or at least 100 regions and at most 1,000 regions.

Example 115. The system of example 87, wherein the one or more processors comprise one or more processors of a smartphone, tablet, laptop, desktop, server, cloud computing architecture, or any combination thereof.

General Method of Converting Image Data into Biomarker Indications

Example 116. A method of determining the presence of a biomarker of a biological sample, comprising: (a) providing a section of a biological sample, wherein the section of the biological sample has been stained; (b) imaging one or more regions of the stained section of the biological sample thereby generating a plurality of images of the stained section; (c) determining the presence of a biomarker of the biological sample as an output of a trained predictive model when the trained predictive model is provided the plurality of images of the stained section an input, wherein the trained predictive model provides an accuracy of determining the presence of the biomarker of at least 80% as compared to genomic sequencing.

Example 117. The method of example 116, wherein the accuracy comprises at least 85%, at least 92%, at least 95%, at least 97%, or at least 99% as compared to genomic sequencing.

Example 118. The method of example 116, wherein the trained predictive model comprises a first predictive model trained on a first plurality of images acquired at a first resolution and a second predictive model trained on a second plurality of images acquired at a second resolution.

Example 119. The method of example 116, wherein the biomarker comprises loss of chromosome 9p.

Example 120. The method of example 116, wherein the biomarker comprises presence of clustered mutations in the gene TP53.

Example 121. The method of example 116, wherein the biomarker comprises presence of clustered mutations in the gene EGFR.

Example 122. The method of example 116, wherein the biomarker comprises presence of clustered mutations in the gene BRAF.

Example 123. The method of example 116, wherein the biomarker comprises presence of clustered mutations in the gene KIT.

Example 124. The method of example 16, wherein the biomarker comprises presence of MSI (vs MSS) and/or MMR gene (e.g., POLE, MLH1, MLH3, MGMT, MSH6, MSH3, MSH2, PMS1, or PMS2) defects.

Example 125. The method of example 116, wherein the biomarker comprises presence of hypermutator mutational signatures: POLE comprised of “POLE” and SBS6, SBS14, SBS15, SBS20, SBS21, SBS2, SBS26, SBS44.

Example 126. The method of example 116, wherein the biomarker comprises presence of high tumor mutational burden.

Example 127. The method of example 116, wherein the biomarker comprises presence of APOBEC alterations and mutational signature.

Example 128. The method of example 116, wherein the biomarker comprises presence of homologous recombination deficiency (HRD). Two commercial HRD companion diagnostic (CDx) tests, Myriad myChoice® CDx and FoundationOne® CDx, have been FDA approved to determine HRD by quantifying overall genomic instability in combination with BRCA1 and BRCA2 status, and, at least three academic HRD detection approaches—SigMA, HRDetect, and CHORD—exist.

Example 129. The method of example 116, wherein the biomarker comprises presence of HRD negative (or homologous recombination proficiency, HRP) or HRD positive, for example, using genomic tests in Example 14.

Example 130. The method of example 116, wherein the biomarker comprises presence of BRCA-1 and/or -2 mutations.

Example 131. The method of example 116, wherein the biomarker comprises presence of “COSMIC3-BRCA” mutational signature, comprising a specific pattern of genome-wide somatic single nucleotide variations (SNVs) defined as “mutational signature 3” (Sig3) in the COSMIC signature catalog, or the presence of genomic ‘scar’ signatures.

Example 132. The method of example 116, wherein the biomarker comprises presence of genomic instability score (GIS), comprised of patterns (or signatures) of loss of heterozygosity (LOH), which are regions of intermediate size; number of telomeric imbalances (telomeric allelic imbalance, or TAI); and large-scale state transitions (LST), which are chromosome breaks (deletions, translocations, and inversions).

Example 133. The method of example 116, wherein the biomarker comprises presence of a homologous recombination feature set which comprises: a total number and proportions of deletions at microhomologies features of the sequencing data, a total number and proportions of genomic segments with loss of heterozygosity features of the sequencing data, a total number and proportions of heterozygous genomic segments features of the sequencing data, a total number and proportions of C:G>T:A single base substitutions at a 5′-NpCpG-3′ contexts features of the sequencing data, or any combination thereof.

Example 134. The method of example 116, wherein the biomarker comprises presence of genomic alterations in one or more of the following homologous recombination repair (HRR)-related or -associated genes beyond BRCA1, BRCA2 (also called ‘BRCA-ness’): alterations in PALB2, BARD1, ATM, BRIP1, CHEK1/2, CDK12, ATR, ATRX, BAP1, ARIDIA, FANC genes (FANCA/C/D2/E/F/G/I/L/M, FANC1), RAD50, RAD51 genes (RAD51 B/C/D/L1/3), RAD52, RAD54L/C/D/B; as well as other less common HRR gene alterations in PPP2R2A, MRE11, MRE11 A, NBN, TP53, NCOR1, PTK2, BLM, WRN, RPA1, EMSY, CCNE1, ERCC3, TAD54, XRCC2/3, HDAC2, NPM1, PTWN, H2AX, RPA; PRK2, NF1.

Example 135. The method of example 116, wherein the biomarker comprises presence of potentially actionable genomic alterations in one or more of the following genes: ABL1, AKT1, APC, ALK, APC, BRAF, RET, ROS, KRAS, NRAS, HRAS, RAF1, KDR, MET, NTRK, NTRK1/2/3, CCNE, CCNE1, CDK4/6, CCND1/2, AR, PDGFRA, PIK3CA, PTEN, CDH1, CDKN2A, CSFIR, CTNNB1, DDR2, DNMT3A, EGFR, ERBB2, ERBB3, ERBB4, HER2/NEU, EZH2, FBXW7, FGF, FGFR, FGFR1, FGFR2, FGFR3, FLT3, FOXL2, GNA11, GNA13, GNAQ, GNAS, HNFIA, MLH1, MPL, MSH6, NOTCH1, VEGFA, HGF, NPM1, PTPN11, RB1, SMAD4, SMARCB1, SMO, SRC, STK11, TP53, TSC1, VHL, ESR1, MAPK3K1, GATA3, CDH1, FBXW7, NF1, KMT2C, CTNNB1, RRAS2, KIFIA, KIF5B, IDH1/2, JAK1/2/3, MAP2K1, MAP3K1, GATA3, PTPN11, SRC, SETBP1, FAT1, KEAP1, LRPIB, FAT3, NF1, RB.

Example 136. The method of example 116, wherein the section of the biological sample comprises a paraffin embedded section, a formalin fixed section, a frozen section, a fresh section, or any combination thereof sections.

Example 137. The method of example 116, wherein the trained predictive model comprises a convolutional neural network.

Example 138. The method of example 116, wherein the trained predictive model comprises a ResNet model.

Example 139. The method of example 116, further comprising reducing a parameter space of the first and second plurality of image data, thereby producing a reduced first and second plurality of image data.

Example 140. The method of example 116, wherein reducing is completed by principal component analysis.

Example 141. The method of example 116, wherein the biological sample comprises a cancer free, or cancerous biological sample.

Example 142. The method of example 116, wherein the biological sample comprises healthy tissue, unhealthy tissue, or any combination thereof tissues.

Example 143. The method of example 142, wherein the unhealthy tissue comprises virally infected tissue.

Example 144. The method of example 143, wherein the virally infected tissue comprises human papilloma virus (HPV) positive tissue.

Example 145. The method of example 144, wherein the virally infected tissue comprises Epstein-Barr virus (EBV), Hepatitis B virus (HBV), Hepatitis C virus (HCV), Human immunodeficiency virus (HIV), Human herpes virus 8 (HHV-8), and/or Human T-cell leukemia virus type, also called human T-lymphotrophic virus (HTLV-1).

Example 146. The method of example 143, wherein the unhealthy tissue comprises nuclei morphology different from nuclei morphology of healthy tissue.

Example 147. The method of example 143, wherein the unhealthy tissue comprises premalignant or precancerous tissue.

Example 148. The method of example 116, wherein the stain comprises a hematoxylin and eosin stain.

Example 149. The method of example 118, wherein the first resolution comprises a 5× magnification, and wherein the second resolution comprises a 20× magnification.

Example 150. The method of example 143, further comprising clustering the reduced first and second plurality of image data thereby generating a first and second clustered dataset.

Example 151. The method of example 150, wherein clustering is completed by k-means clustering.

Example 152. The method of example 150, wherein the trained predictive model is trained with the biological sample's first and second clustered dataset and corresponding biomarker label of the biological sample, wherein the first and second clustered dataset comprise clustered datasets with silhouette coefficients within the top 50th percentile across all clusters of the first and second clustered dataset.

Example 153. The method of example 152, wherein the corresponding biomarker label of the biological sample is determined by genomic sequencing.

Example 154. The method of example 116, wherein the output of the trained predictive model comprises an averaged predicted probability score of the first and second predictive model.

Example 155. The method of example 116, wherein the one or more regions comprise at least 100 regions.

Example 156. The method of example 116, wherein the one or more regions comprise at most 1,000 regions.

Example 157. The method of example 125, further comprising removing one or more nodes of the trained predictive model when provided as an input the reduced first and second plurality of image data.

### Treatment Method for Treating Cancer in a Subject

Example 158. A treatment method for treating cancer in a subject in need thereof, the method comprising: providing a section of a biological sample, wherein the section of the biological sample has been stained; imaging one or more regions of the stained section of the biological sample thereby generating a plurality of images of the stained section; determining the presence of a biomarker of the biological sample as an output of a trained predictive model when the trained predictive model is provided the plurality of images of the stained section an input, wherein the trained predictive model provides an accuracy of determining the presence of the biomarker of at least 80% as compared to genomic sequencing; and administering treatment to the patient based on the presence of the biomarker.

Example 159. The treatment method of example 158, wherein the biomarker comprises loss of chromosome 9p.

Example 160. The treatment method of example 158, wherein the biomarker comprises presence of clustered mutations in the gene TP53.

Example 161. The treatment method of example 158, wherein the biomarker comprises presence of clustered mutations in the gene EGFR.

Example 162. The treatment method of example 158, wherein the biomarker comprises presence of clustered mutations in the gene BRAF.

Example 163. The treatment method of example 158, wherein the biomarker comprises presence of clustered mutations in the gene KIT.

Example 164. The treatment method of example 158, wherein the biomarker comprises presence of MSI (vs MSS) and/or MMR gene (e.g., POLE, MLH1, MLH3, MGMT, MSH6, MSH3, MSH2, PMS1, or PMS2) defects.

Example 165. The treatment method of example 158, wherein the biomarker comprises presence of hypermutator mutational signatures selected from: POLE comprised of “POLE” and SBS6, SBS14, SBS15, SBS20, SBS21, SBS2, SBS26, SBS44.

Example 166. The treatment method of example 158, wherein the biomarker comprises presence of high tumor mutational burden.

Example 167. The treatment method of example 158, wherein the biomarker comprises presence of APOBEC alterations and mutational signature.

Example 168. The treatment method of example 158, wherein the biomarker comprises presence of homologous recombination deficiency (HRD). Two commercial HRD companion diagnostic (CDx) tests, Myriad myChoice® CDx and Foundation One® CDx, have been FDA approved to determine HRD by quantifying overall genomic instability in combination with BRCA1 and BRCA2 status, and, at least three academic HRD detection approaches—SigMA, HRDetect, and CHORD—exist.

Example 169. The treatment method of example 158, wherein the biomarker comprises presence of HRD negative (or homologous recombination proficiency, HRP) or HRD positive, for example, using genomic tests in Example 168.

Example 170. The treatment method of example 158, wherein the biomarker comprises presence of BRCA-1 and/or -2 mutations.

Example 171. The treatment method of example 158, wherein the biomarker comprises presence of “COSMIC3-BRCA” mutational signature, comprising a specific pattern of genome-wide somatic single nucleotide variations (SNVs) defined as “mutational signature 3” (Sig3) in the COSMIC signature catalog, or the presence of genomic ‘scar’ signatures.

Example 172. The treatment method of example 158, wherein the biomarker comprises presence of genomic instability score (GIS), comprised of patterns (or signatures) of loss of heterozygosity (LOH), which are regions of intermediate size; number of telomeric imbalances (telomeric allelic imbalance, or TAI); and large-scale state transitions (LST), which are chromosome breaks (deletions, translocations, and inversions).

Example 173. The treatment method of example 158, wherein the biomarker comprises presence of a homologous recombination feature set which comprises: a total number and proportions of deletions at microhomologies features of the sequencing data, a total number and proportions of genomic segments with loss of heterozygosity features of the sequencing data, a total number and proportions of heterozygous genomic segments features of the sequencing data, a total number and proportions of C:G>T:A single base substitutions at a 5′-NpCpG-3′ contexts features of the sequencing data, or any combination thereof.

Example 174. The treatment method of example 158, wherein the biomarker comprises presence of genomic alterations in one or more of the following homologous recombination repair (HRR)-related or -associated genes beyond BRCA1, BRCA2 (also called ‘BRCA-ness’): alterations in PALB2, BARD1, ATM, BRIP1, CHEK 1/2, CDK12, ATR, ATRX, BAP1, ARIDIA, FANC genes (FANCA/C/D2/E/F/G/I/L/M, FANC1), RAD50, RAD51 genes (RAD51 B/C/D/L1/3), RAD52, RAD54L/C/D/B; as well as other less common HRR gene alterations in PPP2R2A, MRE11, MRE11 A, NBN, TP53, NCOR1, PTK2, BLM, WRN, RPA1, EMSY, CCNE1, ERCC3, TAD54, XRCC2/3, HDAC2, NPM1, PTWN, H2AX, RPA; PRK2, NF1.

Example 175. The treatment method of example 158, wherein the biomarker comprises presence of actionable genomic alterations in one or more of the following genes: ABL1, AKT1, APC, ALK, APC, BRAF, RET, ROS, KRAS, NRAS, HRAS, RAF1, KDR, MET, NTRK, NTRK1/2/3, CCNE, CCNE1, CDK4/6, CCND1/2, AR, PDGFRA, PIK3CA, PTEN, CDH1, CDKN2A, CSFIR, CTNNB1, DDR2, DNMT3A, EGFR, ERBB2, ERBB3, ERBB4, HER2/NEU, EZH2, FBXW7, FGF, FGFR, FGFR1, FGFR2, FGFR3, FLT3, FOXL2, GNA11, GNA13, GNAQ, GNAS, HNFIA, MLH1, MPL, MSH6, NOTCH1, VEGFA, HGF, NPM1, PTPN11, RB1, SMAD4, SMARCB1, SMO, SRC, STK11, TP53, TSC1, VHL, ESR1, MAPK3K1, GATA3, CDH1, FBXW7, NF1, KMT2C, CTNNB1, RRAS2, KIFIA, KIF5B, IDH1/2, JAK1/2/3, MAP2K1, MAP3K1, GATA3, PTPN11, SRC, SETBP1, FAT1, KEAP1, LRPIB, FAT3, NF1, RB.

Example 176. The treatment method of example 163, wherein the patient has GIST; the treatment method comprising not administering c-Kit inhibitor imatinib.

Example 177. The treatment method of example 163, wherein the patient has GIST or other solid tumor; the treatment method comprising not administering c-Kit inhibitors in addition to imatinib, including Axitinib, Dovitinib, Dasatinib, Motesanib diphosphate, Pazopanib, Sunitinib, Masitinib, Vatalanib, Cabozantinib, Tivozanib, Amuvatinib, Telatinib, Pazopanib, Regorafenib, Ripretinib and Dovitinib.

Example 178. The treatment method of any of examples 164-166, further comprising administering a treatment for the cancer comprising the following drugs classes: immune checkpoint inhibitors (ICIs) and other immunotherapies to said subject if said MSI, MMR gene (e.g., POLE, MLH1, MLH3, MGMT, MSH6, MSH3, MSH2, PMS1, or PMS2) defects, hypermutator mutational signatures (e.g., COSMIC14/15/21/26/6) and/or high TMB of said sample is detected.

Example 179. The treatment method of any of examples 164-166, further comprising administering a treatment for the cancer comprising the following drugs classes: PD-1 inhibitors (e.g., Pembrolizumab, Nivolumab, Cemiplimab, Pidilizumab, Dostarlimab, larotrectinib), PD-L1 inhibitors (e.g., Atezolizumab, Avelumab, Durvalumab), CTLA-4 inhibitors (e.g., Ipilimumab and tremelimumab), LAG-3 inhibitors (e.g., tebotelimab, eftilagimod alpha, Relatlimab), TIM-3 inhibitors (e.g., MBG453, Sym023, TSR-022), other immunomodulator therapies alone or in combination with other ICIs or other drugs to said subject if said MSI, MMR gene (e.g., POLE, MLH1, MLH3, MGMT, MSH6, MSH3, MSH2, PMS1, or PMS2) defects, hypermutator mutational signatures (e.g., COSMIC14/15/21/26/6) and/or high TMB of said sample is detected.

Example 180. The treatment method of any of examples 168-174, further comprising administering a treatment for the cancer comprising the following drug classes: platinum drugs, poly-ADP ribose polymerase (PARP) inhibitors, and/or newer agents such as ATR, Wee1 or CHK, Pol-theta or RAD52 inhibitors to said subject if said HRD or surrogate gene or signature wherein said cancer comprises, breast cancer, ovarian cancer, pancreatic adenocarcinoma, prostate cancer, sarcoma, or any solid tumor or combination thereof cancer. In some implementations, the weights collected from a final DeepHRD model trained to detect HRD in one tissue type or modality can be used to initiate the model weights for another tissue type or modality. All other training procedures can stay the same, thus, allowing the transfer of knowledge from training one tissue type or modality to another. The treatment method can utilize this approach to train an ovarian cancer model by utilizing the prior knowledge from the breast cancer model based on some embodiments of the disclosed technology. This AI algorithm will allow application of this deep learning AI technology for other genomic alterations and other cancer types.

Example 181. The treatment method of any of examples 168-174, further comprising administering a treatment for the cancer comprising platinum drugs, including cisplatin, carboplatin, oxaliplatin, nedaplatin, lobaplatin, heptaplatin or satraplatin alone, or in combination with other drugs, e.g., FOLFOX to said subject if said HRD or surrogate gene or signature thereof of said sample is detected. In some embodiments, the cancer therapeutic causes inter-strand breaks of genomic molecules of the subject's cells, leading to p53-initiated apoptosis.

Example 182. The treatment method of any of examples 168-174, further comprising administering a treatment for the cancer comprising poly-ADP ribose polymerase (PARP) inhibitors, including the four main PARP inhibitors: olaparib (Lynparza), niraparib (Zejula), rucaparib (Rubraca), talazoparib (Talzenna) as well as other PARP inhibitors to said subject if said HRD or surrogate gene or signature thereof of said sample is detected.

Example 183. The treatment method of example 158, wherein the treatment method comprising not administering immune checkpoint inhibitors (ICIs) and other immunotherapies to said subject if said 9p deletions of said sample is detected.

Example 184. The treatment method of example 158, wherein the biomarker comprises presence of EGFR/ErbB1 mutations comprising one or more of L858R, exon19del, and exon 20 alteration.

Example 185. The treatment method of examples 184, further comprising administering to the patient afatinib, dacomitinib, erlotinib, gefitinib, osimertinib [T790], or amivantamib.

Example 186. The treatment method of example 158, wherein the biomarker comprises presence of HER2/ErbB2 Amplification.

Example 187. The treatment of example 186, further comprising administering to the patient traztuzumab, ado-trastuzumab emtansine, lapatinib, margetuximab, neratinib, pertuzumab, tucatinimb, deruxtecab, traztumab deruxtecan, or neratinib.

Example 188. The treatment method of example 158, wherein the biomarker comprises presence of BRAF mutation.

Example 189. The treatment method of example 188, further comprising administering to the patient encorafenib, vemurafenib, dabrafenib, trametinib, or cobimetinib.

Example 190. The treatment method of example 158, wherein the biomarker comprises presence of FGFR1/2/3 fusions.

Example 191. The treatment method of example 190, further comprising administering to the patient erdafitanib, fatibatinib, infigratinib, pemigatinib, dovitinib; lenvatinib, pazopanib, ponatinib, or regorafenib.

Example 192. The treatment method of example 158, wherein the biomarker comprises presence of PDGFRA exon 18 mutations.

Example 193. The treatment method of example 192, further comprising administering to the patient avapritinib or dasatinib.

Example 194. The treatment method of example 158, wherein the biomarker comprises presence of KIT mutations in GIST.

Example 195. The treatment method of example 194, further comprising administering to the patient imatinib, Axitinib, Dovitinib, Dasatinib, Motesanib diphosphate, Pazopanib, Sunitinib, Masitinib, Vatalanib, Cabozantinib, Tivozanib, Amuvatinib, Telatinib, Pazopanib, Regorafenib, Ripretinib and Dovitinib, or sorafenib.

Example 196. The treatment method of example 158, wherein the biomarker comprises presence of NRG1 fusion.

Example 197. The treatment method of example 196, further comprising administering to the patient zenocutinumab or seribantmab,

Example 198. The treatment method of example 158, wherein the biomarker comprises presence of RET fusions.

Example 199. The treatment method of example 198, further comprising administering to the patient pralsetinib, selpercatinib; crizotinib, ceritinib, cabozantinib, or vandetanib.

Example 200. The treatment method of example 158, wherein the biomarker comprises presence of ROS1 fusions.

Example 201. The treatment method of example 200, further comprising administering to the patient crizotinib, or entrectinib.

Example 202. The treatment method of example 158, wherein the biomarker comprises presence of NTRK1/2 or 3 fusions.

Example 203. The treatment method of example 202, further comprising administering to the patiententrectinib, larotrectinib, or repotrectinib.

Example 204. The treatment method of example 158, wherein the biomarker comprises presence of ALK fusions.

Example 205. The treatment method of example 204, further comprising administering to the patient crizotinib, alectinib, brigatinib, ceritinib, or lorlatinib.

Example 206. The treatment method of example 158, wherein the biomarker comprises presence of PIK3CA alterations.

Example 207. The treatment method of example 206, further comprising

administering to the patient alpelisib, temsirolimus, or everolimus.

Example 208. The treatment method of example 158, wherein the biomarker comprises presence of Mtor or TSC1/2 mutations.

Example 209. The treatment method of example 208, further comprising administering to the patient temsirolimus, or everolimas.

Example 210. The treatment method of example 158, wherein the biomarker comprises presence of Akt, or PTEN alterations.

Example 211. The treatment method of example 210, further comprising administering to the patient capivasertib.

Example 212. The treatment method of example 158, wherein the biomarker comprises presence of MET amplification or mutation.

Example 213. The treatment method of example 212, further comprising administering to the patient crizotinib, tepotinib, capmatinib, telisotuzumib, tepotinib, or savolitinib.

Example 214. The treatment method of example 158, wherein the biomarker comprises presence of MEK mutation.

Example 215. The treatment method of example 214, further comprising administering to the patient trametinib, cobimetinib, or selumetinib.

Example 216. The treatment method of example 158, wherein the biomarker comprises presence of NF1/2 alterations.

Example 217. The treatment method of example 216, further comprising administering to the patient trametinib, temsirolimus, everolimus, or selumetinib.

Example 218. The treatment method of example 158, wherein the biomarker comprises presence of STK11 alterations.

Example 219. The treatment method of example 218 comprising administering to the patient dasatinib, everolimus, temsirolimus, or bosutinib.

Example 220. The treatment method of example 158, wherein the biomarker comprises presence of KDR alterations.

Example 221. The treatment method of example 220, further comprising administering to the patient pazopanib, regorafenib, or vandetanib.

Example 222. The treatment method of example 158, wherein the biomarker comprises presence of microsatellite stable (MS) with DNA polymerase-ε (POLE) mutation, CD274 amplification, or 9p24.1 amplicon.

Example 223. The treatment method of example 222, further comprising administer ICIs to the patient.

Example 224. The treatment method of example 158, wherein the biomarker comprises presence of MAP2K alterations.

Example 225. The treatment method of example 224, further comprising administering to the patient trametinib.

Example 226. The treatment method of example 158, wherein the biomarker comprises presence of alterations to CCND2, CDK4, or CDKN2A/B.

Example 227. The treatment method of example 226, further comprising administering to the patient Palbociclib.

Example 228. The treatment method of example 158, wherein the biomarker comprises presence of IDH1 mutation.

Example 229. The treatment method of example 228, further comprising administering to the patient ivosidenib.

Example 230. The treatment method of example 158, wherein the biomarker comprises presence of truncating or oncogenic mutations in B2M, PTEN, JAK1, JAK2, STK11 and EGFR, and/or 9p21 or 9p arm/genetic region loss.

Example 231. The treatment method of example 230, further comprising not administering to the patient an immune checkpoint inhibitor.

Example 232. The treatment method of example 158, wherein the biomarker comprises presence of mutations in the RAS genes KRAS and NRAS.

Example 233. The treatment method of example 232, further comprising not administering to the patient epidermal growth factor receptor (EGFR) therapies, like cetuximab and panitumumab, in colorectal cancer, and EGFR tyrosine kinase inhibitors, like erlotinib, in lung cancer.

In some implementations of the disclosed technology, genomic sequencing encompasses any type of genomic profiling where DNA and RNA are subjected to next-generation massively parallel sequencing protocol or genotyping through microarray hybridization.

In some implementations of the disclosed technology, accuracy encompasses the mathematical terms: sensitivity, specificity, precision, negative predictive values, accuracy, and balanced accuracy, or any combination thereof mathematical terms.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A method of determining a presence of a biomarker in a biological sample, comprising:
obtaining a section of a biological sample, wherein the section of the biological sample has been treated with a stain;
imaging one or more regions of the stained section of the biological sample at a first resolution and a second resolution to generate a first and second plurality of image data;
reducing a parameter space of the first and second plurality of image data to produce a reduced first and second plurality of image data; and
providing the first and the second plurality of image data to a trained predictive neural network and determining the presence of a biomarker in the biological sample as an output of the trained predictive neural network.

2. The method of claim 1, wherein the trained predictive neural network is configured to determine the presence of the biomarker with a preset accuracy, wherein the preset accuracy is at least 80% of an accuracy of genomic sequencing.

3. (canceled)

4. The method of claim 1, wherein the trained predictive neural network comprises a first predictive model trained on the first plurality of image data and a second predictive model trained on the second plurality of image data.

5. The method of claim 1, wherein the biomarker comprises loss of chromosome 9p.

6.-9. (canceled)

10. The method of claim 1, wherein the biomarker comprises a presence of at least one of a microsatellite instable (MSI) defect or a mismatch repair (MMR) gene defect, wherein the MMR gene defect includes at least one of POLE, MLH1, MLH3, MGMT, MSH6, MSH3, MSH2, PMS1, or PMS2.

11. The method of claim 1, wherein the biomarker comprises a presence of high tumor mutational burden.

12. The method of claim 1, wherein the biomarker comprises a presence of hypermutator mutational signatures selected from: POLE including POLE and MSI-COSMIC14; MSI combined MSI-COSMIC15, MSI-COSMIC20, MSI-COSMIC21, MSI-COSMIC26, and MSI-COSMIC6.

13. (canceled)

14. The method of claim 1, wherein the biomarker comprises a presence of homologous recombination deficiency (HRD).

15. The method of claim 1, wherein the biomarker comprises a presence of HRD negative or homologous recombination proficiency (HRP) or HRD positive.

16. The method of claim 1, wherein the biomarker comprises a presence of at least one of breast cancer gene (BRCA)-1 mutation or BRCA-2 mutation.

17. (canceled)

18. The method of claim 1, wherein the biomarker comprises a presence of a genomic instability score (GIS) including one or more of: patterns or signatures of loss of heterozygosity (LOH); a number of telomeric imbalances corresponding to a number of regions with allelic imbalance that extend to a sub-telomere but not across a centromere; or large-scale state transitions (LST) corresponding to chromosome breaks, wherein the telomeric imbalances include telomeric allelic imbalances (TAI), wherein the chromosome breaks include deletions, translocations, and inversions.

19. (canceled)

20. (canceled)

21. The method of claim 1, wherein the biomarker comprises a presence of potentially actionable genomic alterations, including in at least one of: ALK, BRAF, RET, ROS, KRAS, NRAS, HRAS, JAK1/2/3, KDR, KIT, MAPK, MTAP, MET, NTRK, NTRK1, PDGFRA, PIK3CA, EGFR, ERBB2, ERBB3, ERBB4, HER2/NEU, FGFR, FGFR1, FGFR2, FGFR3, FLT3, or NRG1.

22. The method of claim 1, wherein the biomarker indicates at least one of immunohistochemical alterations, and copy number alterations, deletions, amplifications, fusions, mutation clusters, mutation signatures or any combination thereof a genome of the biological sample.

23. The method of claim 1, wherein the section of the biological sample comprises a paraffin embedded section, a formalin fixed section, a frozen section, a fresh section, or a combination thereof.

24. The method of claim 1, wherein the trained predictive neural network comprises a convolutional neural network.

25. (canceled)

26. The method of claim 1, wherein the parameter space of the first and second plurality of image data indicates tiles at 5× magnification, wherein the parameter space of the first and second plurality of image data is reduced to 25%, 10%, or 5% of the tiles carrying predictive information.

27. The method of claim 26, wherein reducing is completed by principal component analysis.

28. (canceled)

29. The method of claim 1, wherein the biological sample comprises healthy tissue, unhealthy tissue, or any combination thereof tissues.

30. (canceled)

31. (canceled)

32. The method of claim 29, wherein the unhealthy tissue includes virally infected tissue that comprises one or more of Epstein-Barr virus (EBV), Hepatitis B virus (HBV), Hepatitis C virus (HCV), Human immunodeficiency virus (HIV), Human herpes virus 8 (HHV-8), and/or Human T-cell leukemia virus type corresponding to human T-lymphotrophic virus (HTLV-1).

33. (canceled)

34. (canceled)

35. The method of claim 1, wherein the stain comprises a hematoxylin and eosin stain.

36. The method of claim 1, wherein the first resolution comprises a low magnification of 5× magnification or 10× magnification, and wherein the second resolution comprises a high magnification of 20× magnification or 40× magnification.

37. The method of claim 26, further comprising clustering the reduced first and second plurality of image data to generate a first and second clustered dataset to perform training that produces the trained predictive neural network.

38. The method of claim 37, wherein clustering is completed by k-means clustering.

39. The method of claim 37, wherein the trained predictive neural network is trained with the clustered datasets that represent the top 15% of a variance between clustered datasets of the first and second clustered datasets and corresponding biomarker labels.

40. The method of claim 37, wherein the trained predictive neural network is trained with the first and second clustered dataset and corresponding biomarker label of the biological sample, wherein the first and second clustered dataset comprise clustered datasets with silhouette coefficients within the top 50th percentile across all clusters of the first and second clustered dataset.

41. The method of claim 40, wherein the corresponding biomarker label of the biological sample is determined by genomic sequencing.

42. The method of claim 1, wherein the output of the trained predictive neural network comprises an averaged predicted probability score of the first and second predictive neural network.

43. The method of claim 1, wherein the one or more regions comprise at least 100 regions and at most 10,000 regions.

44. (canceled)

45. The method of claim 1, comprising removing one or more nodes of the trained predictive neural network when the trained predictive neural network is provided an input of the reduced first and second plurality of image data.

46.-86. (canceled)

87. A computer system configured to determine a presence of a biomarker in a biological sample, comprising:
one or more processors; and
a non-transitory computer readable storage medium including software stored thereon, wherein the software comprises executable instructions that, as a result of execution, cause the one or more processors of the computer system to:
obtain a first plurality of image data and a second plurality of image data corresponding to a first set of images and a second set of images of one or more regions of a stained section of a biological sample imaged at a first resolution and at a second resolution, respectively;
reduce a parameter space of the first plurality of image data and the second plurality of image data to produce a reduced first plurality of image data and a second plurality of image data, respectively;
providing the first and the second plurality of image data to a trained predictive model; and
determining the presence of a biomarker in the biological sample as an output of the trained predictive model.

88. The system of claim 87, wherein the trained predictive model is configured to determine the presence of a biomarker with a preset accuracy, wherein the preset accuracy is at least 80% of an accuracy of genomic sequencing.

89. (canceled)

90. The system of claim 87, wherein the trained predictive model comprises a first predictive model trained on the first plurality of image data and a second predictive model trained on the second plurality of image data.

91.-95. (canceled)

96. The system of claim 87, wherein the trained predictive model comprises a convolutional neural network.

97. (canceled)

98. The system of claim 87, wherein the computer system is configured to reduce the parameter space by principal component analysis.

99.-106. (canceled)

107. The system of claim 87, wherein the first resolution comprises a 5× magnification, and wherein the second resolution comprises a 20× magnification.

108. The system of claim 87, wherein the software further comprise instructions that cause the one or more processors of the computer system to cluster the reduced first and second plurality of image data to generate a first and second clustered dataset.

109. The system of claim 108, wherein clustering the reduced first and second plurality of image data is completed by k-means clustering.

110. The system of claim 108, wherein the trained predictive model is trained with the clustered datasets that represent the top 15% of the variance between clustered datasets of the first and second clustered datasets and corresponding biomarker labels.

111. The system of claim 108, wherein the trained predictive model is trained with first and second clustered dataset of the biological sample and corresponding biomarker labels of the biological sample, wherein the first and second clustered dataset comprise clustered datasets with silhouette coefficients within the top 50th percentile across all clusters of the first and second clustered dataset.

112. The system of claim 111, wherein the corresponding biomarker label of the biological sample is determined by genomic sequencing.

113. The system of claim 87, wherein the output of the trained predictive model comprises an averaged predicted probability score of the first and second predictive model.

114. The system of claim 87, wherein the one or more regions comprise at least 100 regions, or at most 1,000 regions, or at least 100 regions and at most 1,000 regions.

115. The system of claim 87, wherein the one or more processors comprise one or more processors of a smart phone, tablet, laptop, desktop, server, cloud computing architecture, or any combination thereof.

116.-233. (canceled)
